(19) United States
(12) Patent Application Publication
(10) Pub. No.: US 2022/0347194 A1
(43) Pub. Date: Nov. 3, 2022

(54) METHODS AND COMPOSITIONS FOR TREATING PAIN AND OTHER EPH RECEPTOR-ASSOCIATED CONDITIONS

(71) Applicant: THE BOARD OF REGENTS OF THE UNIVERSITY OF TEXAS SYSTEM, Austin, TX (US)

(72) Inventors: MAHMOUD SALMA AHMED, DALLAS, TX (US); ENAS KANDIL, Dallas, TX (US); HESHAM SADEK, Dallas, TX (US); PING WANG, Dallas, TX (US); ROBERT BACHOO, Dallas, TX (US); MARK HENKEMEYER, Dallas, TX (US)

(21) Appl. No.: 17/761,014

(22) PCT Filed: Sep. 18, 2020
(86) PCT No.: PCT/US2020/070557

§ 371 Date: Mar. 16, 2022

Related U.S. Application Data
(60) Provisional application No. 62/902,135, filed on Sep. 18, 2019.

(51) Int. Cl. A61K 31/65; A61K 31/167; A61K 31/165; A61K 31/045; A61K 31/195; A61P 29/00; A61P 35/00; A61K 45/06

(52) U.S. Cl. CPC A61K 31/65; A61K 31/167; A61K 31/165; A61K 31/045; A61K 31/195; A61P 29/00; A61P 35/00; A61K 45/06

(57) ABSTRACT

Inhibition of EphB receptors (e.g., EphB1) can be used in therapeutic methods for treating EphB receptor-associated conditions (e.g., pain, cancer). Demeclocycline, chlortetracycline, and minocycline are identified as EphB receptor inhibitors. Accordingly, aspects of the disclosure relate to methods for treating pain comprising providing demeclocycline, chlortetracycline, minocycline, or derivatives thereof, alone or in combination, to an individual in need thereof. Further aspects relate to pharmaceutical compositions comprising two or more of minocycline, demeclocycline, chlortetracycline, and/or derivatives thereof.

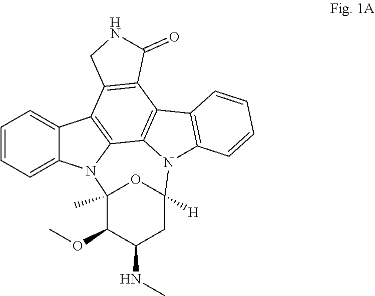 [SMILES: CN[C@@H]1C[C@H]2O[C@@](C)([C@@H]1OC)n1c3ccccc3c3c4c(c5c6ccccc6n2c5c31)C(=O)NC4]

This application claims the benefit of priority of U.S. Provisional Patent Application No. 62/902,135 filed Sep. 18, 2019, which is hereby incorporated by reference in its entirety.

### BACKGROUND

### Field of the Invention

This invention relates to the field of molecular biology and medicine.

### Background

The family of Eph (erythropoietin-producing hepatocellular carcinoma) receptor tyrosine kinases have been implicated in multiple different clinical problems, including neurological disorders like Alzheimer's disease, anxiety, and neuropathic pain, malignancies, fibrotic diseases, and viral infections (1-4). Eph receptors are highly conserved molecules that are grouped into two subfamilies of nine EphA and five EphB receptors based on sequence similarity and whether they promiscuously bind GPI-anchored ligands (ephrin-A) or transmembrane ligands (ephrin-B), respectively (5). As both Eph receptors and ephrins are membrane-anchored, receptor-ligand interactions generally occur upon cell-cell contact, and this leads to the transduction of bidirectional intracellular signals into both the Eph-expressing cell and ephrin-expressing cell (6).

The Eph-ephrin interaction has been targeted for drug discovery using high-throughput approaches to identify peptides and small molecular weight chemicals (7, 8). These compounds dock into a deep pocket formed in the N-terminal globular ephrin-binding domain of the Eph receptor ectodomain within the G-H loop and disrupt protein-protein interfaces involved in receptor-ligand dimerization. While small molecules have the ability to disrupt the Eph-ephrin interaction, this approach is challenging as compounds may have short biological half-life and, due to their limited size, small compounds may lack robust binding affinities to compete with the large receptor-ligand interface formed upon Eph-ephrin binding (9).

Despite their high relevance to a wide range of diseases, there are currently no approved FDA drugs targeting any of the Eph receptors. There is a need in the art for molecules capable of targeting Eph receptors for use as therapy (e.g., treating pain).

### SUMMARY OF THE DISCLOSURE

The present disclosure is based, at least in part, on the identification of compounds capable of binding to and inhibiting EphB1, EphB2, and EphB4 receptors and treating or preventing pain. Accordingly, aspects of the disclosure relate to a method of treating pain in a subject comprising administering to the subject one or more compounds selected from demeclocycline, chlortetracycline, and derivatives thereof. Further aspects of the disclosure relate to a pharmaceutical composition comprising (a) minocycline or a derivative thereof; and (b) a compound selected from demeclocycline, chlortetracycline, and derivatives thereof.

Further aspects of the disclosure are directed to a method of treating an Eph receptor-associated condition.

Further aspects of the disclosure relate to pharmaceutical compositions comprising: (a) minocycline or a derivative thereof; and (b) a compound selected from demeclocycline, chlortetracycline, and derivatives thereof.

Embodiments of the present disclosure include pharmaceutical compositions and uses thereof, kits and uses thereof, and various methods, including methods of inhibiting an EphB enzyme, methods of treating pain, methods of reducing opioid dependence, methods of alleviating opioid withdrawal symptoms, methods of treating opioid withdrawal pain, methods of treating an EphB receptor-associated condition, methods of treating a subject for cancer, methods of treating a subject for a neurological disorder, and methods of treating a subject for a viral infection.

Compositions of the present disclosure can include at least 1, 2, 3, or more of the following components: minocycline, a derivative of minocycline, demeclocycline, a derivative of demeclocycline, chlortetracycline, a derivative of chlortetracycline, an EphB1 inhibitor, an EphB2 inhibitor, an EphB4 inhibitor, an analgesic, an anticonvulsant, an antidepressant, a chemotherapeutic, an immunotherapeutic, a cancer therapeutic, and an excipient. Any one or more of these components may be excluded from certain embodiments of the disclosure.

Methods of the present disclosure can include at least 1, 2, 3, or more of the following steps: administering minocycline, administering a derivative of minocycline, administering demeclocycline, administering a derivative of demeclocycline, administering chlortetracycline, administering a derivative of chlortetracycline, administering an EphB1 inhibitor, administering an EphB2 inhibitor, administering an EphB4 inhibitor, administering an analgesic, administering an anticonvulsant, administering an antidepressant, administering a chemotherapeutic, administering an immunotherapeutic, administering a cancer therapeutic, inhibiting an EphB receptor, and diagnosing a subject for an EphB receptor-associated condition. Any one or more of these steps may be excluded from certain embodiments of the disclosure.

In some embodiments, disclosed herein is a method of treating pain in a subject comprising administering to the subject one or more compounds selected from demeclocycline, chlortetracycline, and derivatives thereof. In some embodiments, demeclocycline is administered to the subject. In some embodiments, chlortetracycline is administered to the subject. In some embodiments, the method further comprises administering minocycline or a derivative thereof to the subject. In some embodiments, the pain is chronic pain, acute pain, neuropathic pain, radicular pain associated with degenerative disc disease, diabetic neuropathy pain, complex regional pain syndrome, peripheral ischemic neuropathy pain, small fiber neuropathy pain, large fiber neuropathy pain, neuropathy associated with neurofibromatosis, post herpetic neuralgia pain, ilioinguinal neuralgia, anterior abdominal wall entrapment syndrome, lateral femerocutaneous neuralgia, cancer associated pain, somatic pain, visceral pain, or opioid withdrawal pain. In some embodiments, the pain excludes one or more of neuropathic pain, radicular pain associated with degenerative disc disease, diabetic neuropathy pain, complex regional pain syndrome, peripheral ischemic neuropathy pain, small fiber neuropathy pain, large fiber neuropathy pain, neuropathy associated with neurofibromatosis, post herpetic neuralgia pain, ilioinguinal neuralgia, anterior abdominal wall entrapment syndrome, lateral femerocutaneous neuralgia, cancer associated pain, somatic pain, and visceral pain. In some embodiments, the pain is opioid withdrawal pain. In some embodiments, the pain excludes opioid withdrawal pain. In some embodiments, the compound prevents the pain. In some embodiments, the compound reduces the severity of the pain. In some embodiments, the one or more compounds are administered prior to surgery or prior to opioid cessation. In some embodiments, the one or more compounds are administered immediately after a surgical procedure or opioid cessation.

In some embodiments, the Eph receptor-associated condition is a cancer. In some embodiments, the cancer is a recurrent cancer. In some embodiments, the cancer is a metastatic cancer. In some embodiments, the cancer is a Stage I cancer, a Stage II cancer, a Stage III cancer, or a Stage IV cancer. In some embodiments, the cancer is brain cancer. In some embodiments, the cancer is a grade I cancer, a grade II cancer, a grade III cancer, or a grade IV cancer. In some embodiments, the brain cancer is glioblastoma. In some embodiments, the Eph receptor-associated condition is a neurological disorder. In some embodiments, the Eph receptor-associated condition is a fibrotic disease. In some embodiments, the Eph receptor-associated condition is a viral infection.

In some embodiments, the method comprises administering to the subject at least two compounds selected from demeclocycline, chlortetracycline, minocycline, and derivatives thereof. In some embodiments, the at least two compounds are administered substantially simultaneously. In some embodiments, the at least two compounds are administered sequentially. The at least two compounds may be in the same composition or in separate compositions. In some embodiments, the IC₅₀ of the at least two compounds against an EphB enzyme in the subject is reduced relative to the IC₅₀ of a single compound of the at least two compounds. In some embodiments, the IC₅₀ of at least one of the compounds, when administered with an additional compound, is reduced by at least 10, 20, 30, 40, 50, or 60% relative to the administration of the single compound alone. In some embodiments, an administered dose of the at least two compounds is reduced compared to a standard administered dose of a single compound. In some embodiments, an administered dose of at least one of the compounds, when administered with an additional compound, is reduced by at least 5, 10, 15, 20, 25, 30, 35, 40, 45, 50, 55, or 60% relative to the administration of the single compound alone. In some embodiments, the method comprises administering to the subject at least three compounds selected from demeclocycline, chlortetracycline, minocycline, and derivatives thereof. In some embodiments, the at least three compounds are administered substantially simultaneously. In some embodiments, the at least three compounds are administered sequentially. In some embodiments, the IC₅₀ of the at least three compounds against an EphB enzyme in the subject is reduced relative to the IC₅₀ of a single compound of the at least three compounds. For example, the IC₅₀ of demeclocyline, chlortetracycline, or minocycline, when administered in combination with at least 1 or 2 other compounds of the disclosure, may be reduced by at least 10, 20, 30, 40, 50, or 60% relative to demeclocyline, chlortetracycline, or minocycline, respectively, administered alone. In some embodiments, the method comprises administering to the subject demeclocycline, chlortetracycline, and minocycline. In some embodiments, the demeclocycline, chlortetracycline, and minocycline are administered substantially simultaneously. In some embodiments, the demeclocycline, chlortetracycline, and minocycline are administered sequentially. In some embodiments, the IC₅₀ of the demeclocycline, chlortetracycline, and minocycline against an EphB enzyme in the subject is reduced relative to the IC₅₀ of a demeclocycline, chlortetracycline, or minocycline alone.

In some embodiments, the method further comprises administration of an additional therapeutic agent. In some embodiments, the additional therapeutic agent is an analgesic. In some embodiments, the analgesic is an opioid, a nonsteroidal anti-inflammatory drug (NSAID), acetaminophen, a steroid, a COX-2 inhibitor, a topical analgesic, or any combination thereof. In some embodiments, the topical analgesic is lidocaine, capsaicin, or menthol. In some embodiments, the additional therapeutic is an anticonvulsant. In some embodiments, the anticonvulsant is a gabapentinoid. In some embodiments, the additional therapeutic is an antidepressant. In some embodiments, the antidepressant is a selective serotonin reuptake inhibitor. In some embodiments, the additional agent is one that is described herein. In some embodiments, the additional therapeutic agent is a chemotherapeutic, immunotherapeutic, or other cancer therapeutic.

In some embodiments, the subject does not have a bacterial infection or has not been diagnosed with a current bacterial infection. In some embodiments, the subject is one that is not currently undergoing an antibiotic regimen for a bacterial infection. In some embodiments, the subject has previously been treated for the pain. In some embodiments, the subject has not previously been treated for the pain. In some embodiments, the subject has previously been treated with an additional therapeutic agent. The treatment with an additional therapeutic agent may be one described herein. In some embodiments, the subject has been determined to be unresponsive or have a toxic response to the additional therapeutic agent.

In some embodiments, the method comprises inhibiting an Eph receptor in the subject with the compound. In some embodiments, the compound binds to the catalytic domain of the Eph receptor. In some embodiments, the compound does not disrupt an interaction between the Eph receptor and an ephrin ligand. In some embodiments, the compound is provided at a dose sufficient to reduce the activity of the Eph receptor by at least 50%. In some embodiments, the compound is provided at a dose sufficient to reduce the activity of the Eph receptor by at least 10, 20, 30, 40, 50, 60, or 70%. In some embodiments, the Eph receptor is EphB1, EphB2, EphB3, EphB4, or a variant thereof. In some embodiments, the Eph receptor is EphB1.

In some embodiments, the pharmaceutical composition further comprises a pharmaceutically acceptable excipient. In some embodiments, the pharmaceutical composition comprises an amount of the minocycline or derivative thereof and the compound sufficient to reduce the activity of an Eph receptor by at least 50%. In some embodiments, the pharmaceutical composition comprises an amount of the minocycline or derivative thereof and the compound sufficient to reduce the activity of an Eph receptor by at least 10, 20, 30, 40, 50, 60, or 70%. In some embodiments, the Eph receptor is EphB1, EphB2, EphB3, EphB4, or a variant thereof. In some embodiments, the Eph receptor is EphB1. In some embodiments, the pharmaceutical composition comprises: (a) minocycline or a derivative thereof; (b) demeclocycline or a derivative thereof; and (c) chlortetracycline or a derivative thereof. In some embodiments, the pharmaceutical composition comprises minocycline, demeclocycline, and chlortetracycline. In some embodiments, the pharmaceutical composition further comprises an additional therapeutic agent. In some embodiments, the additional therapeutic agent comprises an analgesic. In some embodiments, the analgesic is an opioid, a nonsteroidal anti-inflammatory drug (NSAID), acetaminophen, a steroid, a COX-2 inhibitor, a topical analgesic, or any combination thereof. In some embodiments, the topical analgesic is lidocaine, capsaicin, or menthol. In some embodiments, the additional therapeutic is an anticonvulsant. In some embodiments, the anticonvulsant is a gabapentinoid. In some embodiments, the additional therapeutic is an antidepressant. In some embodiments, the antidepressant is a selective serotonin reuptake inhibitor. In some embodiments, the additional therapeutic agent is a chemotherapeutic, immunotherapeutic, or other cancer therapeutic. In some embodiments, disclosed herein is a kit comprising a pharmaceutical composition described herein. In some embodiments, the kit further comprises instructions for use.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A shows the structure of staurosporine, selected as the query for molecular similarity simulations. FIG. 1B shows a schematic flow chart for the in silico molecular virtual screening, starting with energy minimized FDA approved small molecules using MMFF94 force field to be checked for degree of superimposition based on Staurosporine. This was followed by selecting the top 100 hits based on their Tanimoto coefficient ratio to undergo semi-flexible docking study using molecular Operating Environment (MOE), to end up with the top 10 drug candidates. FIG. 1C shows a space filling representation for the EphB1 tyrosine kinase domain, where the circle refers to the binding domain of identified small molecules.

FIGS. 2A-2C show 2D chemical structures of demeclocycline, chlortetracycline, and minocycline, and their structural superimposition and along with staurosporine. FIGS. 2E-2G show visual representations of the following molecules docket with the EphB1 kinase domain: demeclocycline (FIG. 2E), showing hydrophobic interaction where dotted lines represent hydrogen bonding along with Met 700:A and Thr 697:A; chlortetracycline (FIG. 3F), where hydrogen bonds situated along with MET 700:A and GLN 711:A, and minocycline (FIG. 3G), where hydrogen bonds along with MET 700:A and GLU 668:A.

FIG. 3 shows IC₅₀ curves for ramelteon, galantamine, oxytetracycline, and darifenacin in an EphB1 protein kinase activity inhibition assay.

FIGS. 4A-4C show IC₅₀ curves for demeclocycline alone (FIG. 4A), minocycline alone (FIG. 4B), and chlortetracycline alone (FIG. 4C), in EphB1 (top left), EphB2 (top right), EphB3 (bottom left), and EphB4 (bottom right) protein kinase activity inhibition assays.

FIGS. 5A and 5B show the results of synergy experiments described in Example 2. FIG. 5A shows IC₅₀ values for the shown combinations of demeclocycline, minocycline, and chlortetracycline in protein kinase activity inhibition assays against EphB1, EphB3, and EphB4.

FIG. 5B shows IC₅₀ curves for minocycline, chlortetracycline, and demeclocycline together (MCD) in protein kinase activity inhibition assays against EphB1 (top left), EphB2 (top right), EphB3 (bottom left), and EphB4 (bottom right).

FIGS. 6A and 6B show the development of thermal hyperalgesia using Hargreaves (FIG. 6A) and mechanical allodynia using von Frey filaments (FIG. 6B) after intra-plantar right paw injection of 0.01% capsaicin for PBS, demeclocycline, demeclocycline and chlortetracycline (DC), and minocycline, demeclocycline and chlortetracycline (MCD) treated mice. The left paw was remained un-injected. ***P<0.001; Significant difference based on the PBS (control) group. One-way ANOVA was conducted; Data is expressed as mean±S.D (n=5).

FIGS. 7A and 7B show the development of thermal hyperalgesia using Hargreaves (FIG. 7A) and mechanical allodynia using von Frey (FIG. 7B) after intra-plantar right paw injection of Complete freund's adjuvant (CFA) for PBS, Demeclocycline, DC, and MCD treated mice. The left paw was un-injected. A repeated measures ANOVA, followed by post hoc tests using the Bonferroni correction was conducted. Data is expressed as mean±S.D (n=5). FIG. 7C shows western blotting and densitometry analysis for PBS, demeclocycline, DC, and MCD treated brain lysates, demonstrating that MCD significantly inhibits the phosphorylation of EphB 1/2.

FIG. 7D shows immunostaining for PBS treated vs MCD treated spinal cords, demonstrating that the phosphorylated form of EphB 1/2 receptors was diminished in the MCD treated spinal cord. Neurons were stained with NeuN and pEphB2 antibodies.

FIG. 8 shows a cartoon representation of the structure of EphB1 bound to chlortetracycline.

FIG. 9A shows a superimposition of apo EphB1, EphB1 bound to chlortetracycline, and EphB1 bound to quinazoline. FIG. 9B shows the overall structure of EphB1 bound to chlortetracycline. FIG. 9C shows a superimposition of the cartoon crystal structure of EphB1 bound to chrlotetracycline and the docking structure of EphB1 bound to chrlotetracycline.

FIGS. 10A-C show example images of untreated mice harboring glioma tumors, as described in Example 5.

FIGS. 11A-C show example images of mice treated with MCD, as described in Example 5.

FIGS. 12A and 12B show H&E staining of mouse brain tissue, as described in example 6. FIG. 12A shows staining of untreated (Vehicle) mice and FIG. 12B show staining of mice treated with MCD.

FIG. 13 shows results from the studies described in Example 8.

### DESCRIPTION OF ILLUSTRATIVE EMBODIMENTS

The intracellular Eph tyrosine kinase catalytic domain provides an alternative portion of the receptor to target for drug discovery, separate from the ephrin-binding domain. Kung et al. recently described an irreversible and specific inhibitor of the EphB3 kinase domain that covalently binds to the Cys717 residue not present in the other Eph receptors (10). The ultimate utility of such a compound remains unclear because little is known about potential involvement of EphB3 in disease states. By contrast, the EphB1, EphB2, and EphB4 receptors have been implicated in numerous processes, and EphB1 is thought to play a critical role in chronic pain (11-23). The inventors identified, using in silico virtual screening, a number of compounds with an ability to bind to and inhibit the EphB1, EphB2, and EphB3 receptors. These compounds, which include demeclocycline, minocycline, and chlortetracycline, are useful as inhibitors of EphB receptors and in treatment of EphB receptor-associated conditions (e.g., pain).

### I. Definitions

“Individual, “subject,” and “patient” are used interchangeably and can refer to a human or non-human.

The terms “lower,” “reduced,” “reduction,” “decrease,” or “inhibit” are all used herein generally to mean a decrease by a statistically significant amount. However, for avoidance of doubt, “lower,” “reduced,” “reduction, “decrease,” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention. With respect to pharmaceutical compositions, the term “consisting essentially of” includes the active ingredients recited, excludes any other active ingredients, but does not exclude any pharmaceutical excipients or other components that are not therapeutically active.

The term “consisting of” refers to compositions, methods, and respective components thereof as described herein, which are exclusive of any element not recited in that description of the embodiment.

As used in this specification and the appended claims, the singular forms “a”, “an”, and “the” include plural references unless the context clearly dictates otherwise. Thus for example, references to “the method” includes one or more methods, and/or steps of the type described herein and/or which will become apparent to those persons skilled in the art upon reading this disclosure and so forth.

### II. EphB Receptor Inhibitors

Aspects of the present disclosure may include compositions comprising one or more EphB receptor inhibitors. An EphB receptor inhibitor may refer to any member of the class of compound or agent having an IC₅₀ of 100 μM or lower concentration for inhibition of activity of an EphB receptor (e.g., EphB1, EphB2, EphB3, EphB4), for example, at least or at most or about 100 μM, 80 μM, 50 μM, 40 μM, 20 μM, 10 μM, 5 μM, 1 μM, 100 nM, 10 nM, 1 nM or lower concentration (or any range or value derivable therefrom) or any compound or agent that inhibits activity of an EphB receptor. An EphB receptor inhibitor may be specific for a single receptor (e.g., EphB1) or may by capable of inhibiting multiple types of EphB receptors. In some embodiments, an EphB receptor is capable of inhibiting a subset of EphB receptor types (e.g., EphB1, EphB3, and EphB4) but not all EphB receptor types. In some embodiments, an EphB receptor is capable of inhibiting all types of EphB receptors. In some embodiments, an EphB receptor inhibitor is an EphB1 receptor inhibitor. In some embodiments, an EphB receptor inhibitor is an EphB2 receptor inhibitor. In some embodiments, an EphB receptor inhibitor is an EphB3 receptor inhibitor. In some embodiments, an EphB receptor inhibitor is an EphB4 receptor inhibitor.

In some embodiments, an EphB receptor inhibitor of the present disclosure is minocycline or a derivative thereof. A minocycline derivative of the disclosure may be any molecule derived from (e.g., chemically derived from) minocycline having the ability to inhibit an EphB receptor. In some embodiments, an EphB receptor inhibitor of the present disclosure is demeclocycline or a derivative thereof. A demeclocycline derivative of the disclosure may be any molecule derived from (e.g., chemically derived from) demeclocycline having the ability to inhibit an EphB receptor. In some embodiments, an EphB receptor inhibitor of the present disclosure is chlortetracycline or a derivative thereof. A chlortetracycline derivative of the disclosure may be any molecule derived from (e.g., chemically derived from) chlortetracycline having the ability to inhibit an EphB receptor. Certain minocycline derivatives, demeclocycline derivatives, and chlortetracycline derivatives are described in, for example, U.S. Patent Application Publication US2016/0030452, U.S. Pat. Nos. 7,696,188, and 9,533,943, incorporated herein by reference in their entirety.

In some embodiments, an EphB receptor inhibitor is a compound or agent capable of binding to a catalytic domain of an EphB receptor. In some embodiments, an EphB receptor inhibitor is a compound or agent which is not capable of binding to an ephrin-binding domain of an EphB receptor. Exemplary EphB receptor inhibitors are disclosed herein and include, for example, demeclocycline, minocycline, and chlortetracycline. Multiple EphB receptor inhibitors may be capable of inhibiting the activity of an EphB receptor in a synergistic fashion, wherein the IC₅₀ of multiple inhibitors against an EphB receptor activity is reduced relative to the IC₅₀ of a single inhibitor. For example, in some embodiments, an IC₅₀ of demeclocycline against an EphB1 receptor is higher than the IC₅₀ of demeclocycline, chlortetracycline, and minocycline together. In this case, a lower dose of multiple EphB receptor inhibitors (e.g., demeclocycline, chlortetracycline, and minocycline) may be used in the disclosed treatment methods (e.g., pain treatment) to obtain an equivalent efficacy as a higher dose of a single EphB receptor inhibitor.

### III. Pharmaceutical Compositions

Methods and compositions may be provided for the treatment of an EphB receptor-associated disorder. In some embodiments, methods and compositions are provided for the treatment of pain. In some embodiments, methods and compositions are provided for the treatment of a brain cancer (e.g., glioblastoma). In certain embodiments, there may be provided methods and compositions involving pharmaceutical compositions that comprise one or more therapeutic agents (e.g., EphB receptor inhibitors) as described herein. In some embodiments, a pharmaceutical composition of the present disclosure comprises minocycline or a derivative thereof and a compound selected from demeclocycline, chlortetracycline, and derivatives thereof. In some embodiments, a pharmaceutical composition of the present disclosure comprises minocycline and demeclocycline. In some embodiments, a pharmaceutical composition of the present disclosure comprises minocycline and chlortetracycline. In some embodiments, a pharmaceutical composition of the present disclosure comprises minocycline, chlortetracycline, and demeclocycline.

The therapeutic agents useful in the methods may be in the form of free acids, free bases, or pharmaceutically acceptable addition salts thereof. Such salts can be readily prepared by treating the agents with an appropriate acid. Such acids include, by way of example and not limitation, inorganic acids such as hydrohalic acids (hydrochloric, hydrobromic, hydrofluoric, etc.), sulfuric acid, nitric acid, and phosphoric acid, and organic acids such as acetic acid, propanoic acid, 2-hydroxyacetic acid, 2-hydroxypropanoic acid, 2-oxopropanoic acid, propandioic acid, and butandioic acid. Conversely, the salt can be converted into the free base form by treatment with alkali.

Aqueous compositions in some aspects comprise an effective amount of the therapeutic agent, further dispersed in pharmaceutically acceptable carrier or aqueous medium. The phrase “pharmaceutically or pharmacologically acceptable” refer to compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the pharmaceutical compositions is contemplated. Supplementary active ingredients also can be incorporated into the compositions.

Solutions of pharmaceutical compositions can be prepared in water suitably mixed with a surfactant, such as hydroxypropylcellulose. Dispersions also can be prepared in glycerol, liquid polyethylene glycols, mixtures thereof, and in oils. Under ordinary conditions of storage and use, these preparations contain a preservative to prevent the growth of microorganisms.

The pharmaceutical compositions may be administered in the form of injectable compositions either as liquid solutions or suspensions; solid forms suitable for solution in, or suspension in, liquid prior to injection may also be prepared. These preparations also may be emulsified. For instance, the composition may contain at least about, at most about, or about 1, 5, 10, 25, 50 mg or up to about 100 mg of human serum albumin per milliliter of phosphate buffered saline. Other pharmaceutically acceptable carriers include aqueous solutions, non-aqueous solvents, non-toxic excipients, including salts, preservatives, buffers and the like.

Examples of non-aqueous solvents include propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well-known parameters.

Administration of pharmaceutical compositions may be via any common route so long as the target tissue, cell or intracellular department is available via that route. This includes oral, nasal, buccal, rectal, vaginal or topical. Alternatively, administration may be by orthotopic, intradermal subcutaneous, intramuscular, intraperitoneal or intravenous injection. Such compositions would normally be administered as pharmaceutically acceptable compositions that include physiologically acceptable carriers, buffers or other excipients. Volume of an aerosol may be between about 0.01 mL and 0.5 mL.

Additional formulations may be suitable for oral administration. “Oral administration” as used herein refers to any form of delivery of a therapeutic agent or composition thereof to a subject wherein the agent or composition is placed in the mouth of the subject, whether or not the agent or composition is swallowed. Thus, “Oral administration” includes buccal and sublingual as well as esophageal administration. Absorption of the agent can occur in any part or parts of the gastrointestinal tract including the mouth, esophagus, stomach, duodenum, ileum and colon. Oral formulations include such typical excipients as, for example, pharmaceutical grades of mannitol, lactose, starch, magnesium stearate, sodium saccharine, cellulose, magnesium carbonate and the like. The compositions may take the form of solutions, suspensions, tablets, pills, capsules, sustained release formulations or powders.

In one embodiment, the oral formulation can comprise the therapeutic agent and one or more bulking agents. Suitable bulking agents are any such agent that is compatible with the therapeutic agent including, for example, lactose, microcrystalline cellulose, and non-reducing sugars, such as mannitol, xylitol, and sorbitol. One example of a suitable oral formulation includes spray-dried therapeutic agent-containing polymer nanoparticles (e.g., spray-dried poly(lactide-co-glycolide)/amifostine nanoparticles having a mean diameter of between about 150 nm and 450 nm; see Pamujula, et al., 2004, which is here by incorporated by reference in its entirety). The nanoparticles can contain between about 20 and 50 w/w % therapeutic agent for example, between about 25% and 50%.

In some embodiments, when the route is topical, the form may be a cream, ointment, salve or spray. Topical formulations may include solvents such as, but not limited to, dimethyl sulfoxide, water, N,N-dimethylformamide, propylene glycol, 2-pyrrolidone, methyl-2-pyrrolidone, and/or N-methylforamide. To enhance skin permeability, if necessary, the skin area to be treated can be pre-treated with dimethylsulfoxide; see Lamperti et al., 1990, which is hereby incorporated by reference in its entirety.

In other embodiments, the pharmaceutical compositions may be for subcutaneous administration (e.g., injection and/or implantation). For example, implantable forms may be useful for patients which are expected to undergo multiple CT scans over an extended period of time (e.g., one week, two weeks, one month, etc.). In one example, such subcutaneous forms can comprise the therapeutic agent and a carrier, such as a polymer. The polymers may be suitable for immediate or extended release depending on the intended use. In one example, the therapeutic agent can be combined with a biodegradable polymer (e.g., polylactide, polyglycolide, and/or a copolymers thereof). In another example, subcutaneous forms can comprise a microencapsulated form of the therapeutic agent, see, e.g., Srinivasan et al., 2002, which is hereby incorporated by reference in its entirety. Such microencapsulated forms may comprise the therapeutic agent and one or more surfactant and other excipients (e.g., lactose, sellulose, cholesterol, and phosphate- and/or stearate-based surfactants).

In a further embodiment, the therapeutic agent or pharmaceutical compositions may be administered transdermally through the use of an adhesive patch that is placed on the skin to deliver the therapeutic agent through the skin and into the bloodstream. An advantage of the transdermal drug delivery route relative to other delivery systems such as oral, topical, or intravenous is that the patch provides a controlled release of the therapeutic agent into the patient, usually through a porous membrane covering a reservoir of the therapeutic agent or through body heat melting thin layers of therapeutic agent embedded in the adhesive. In practicing certain aspects, any suitable transdermal patch system may be used, including, without limitation, single-layer drug-in-adhesive, multi-layer drug-in-adhesive, and reservoir.

An effective amount of the pharmaceutical composition may be determined based on the intended goal, such as treating pain. The term “unit dose” or “dosage” refers to physically discrete units suitable for use in a subject, each unit containing a predetermined quantity of the therapeutic agent calculated to produce the desired responses, discussed above, in association with its administration, i.e., the appropriate route and treatment regimen.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 5 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IV. Methods of Treatment

Compositions of the present disclosure may be administered to any individual who is suffering from a condition associated with an Eph receptor, such as an EphB receptor, where inhibition of an EphB receptor may serve to treat the condition. A condition may be associated with activity or expression of one of more of an EphB1, EphB2, EphB3, or EphB4 receptor. EphB receptor-associated conditions which may be treated using the disclosed compositions include, but are not limited to, neurological disorders (e.g., Alzheimer's disease, anxiety, and pain such as neuropathic pain), malignancies (e.g., cancer, including brain cancer such as glioma), fibrotic diseases, and viral infections. The term “treatment” or “treating” means any treatment of a disease in a mammal, including:

(i) preventing the disease, that is, causing the clinical symptoms of the disease not to develop by administration of a protective composition prior to the induction of the disease;

(ii) suppressing the disease, that is, causing the clinical symptoms of the disease not to develop by administration of a protective composition after the inductive event but prior to the clinical appearance or reappearance of the disease;

(iii) inhibiting the disease, that is, arresting the development of clinical symptoms by administration of a protective composition after their initial appearance; and/or

(iv) relieving the disease, that is, causing the regression of clinical symptoms by administration of a protective composition after their initial appearance.

A. Cancer Treatment

In some embodiments, compositions of the present disclosure are useful in treatment of conditions associated with an Eph receptor. Conditions associated with an Eph receptor include, for example, cancer.

In some embodiments, the disclosed methods comprise administering a cancer therapy to a subject or patient. The cancer therapy may be chosen based on the expression level measurements, alone or in combination with the clinical risk score calculated for the subject. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be a checkpoint inhibitor therapy. In some embodiments, the cancer therapy comprises one or more EphB receptor inhibitors, such as demeclocycline, chlortetracycline, minocycline, and derivatives thereof. Any of these cancer therapies may also be excluded. Combinations of these therapies may also be administered.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, disclosed are methods and compositions for the treatment of a brain cancer (e.g., glioma). In some embodiments, disclosed herein are methods for treatment of brain cancer, such as glioma, comprising providing to an individual in need thereof an effective amount of one or more compounds capable of inhibiting an Eph receptor activity. In some embodiments, the brain cancer is glioma. In some embodiments, the brain cancer is glioblastoma. Compounds capable of inhibiting Eph receptor activity are disclosed herein, and include, for example, demeclocycline, chlortetracycline, and minocycline.

B. Pain Treatment

In some embodiments, compositions of the present disclosure are useful in treatment of conditions associated with an EphB (e.g., EphB1) receptor. Conditions associated with an EphB1 receptor include, for example, pain. In some embodiments, disclosed herein are methods for treatment of pain comprising providing to an individual in need thereof an effective amount of one or more compounds capable of inhibiting EphB1 activity. Compounds capable of inhibiting EphB1 activity are disclosed herein, and include, for example, demeclocycline, chlortetracycline, and minocycline.

In some embodiments, the disclosed methods comprise preventing and/or reducing the severity of pain in an individual. In some embodiments, the pain is chronic pain. In some embodiments, the pain is acute pain. In some embodiments, the pain is neuropathic pain, radicular pain associated with degenerative disc disease, diabetic neuropathy pain, complex regional pain syndrome, peripheral ischemic neuropathy pain, small fiber neuropathy pain, large fiber neuropathy pain, neuropathy associated with neurofibromatosis, post herpetic neuralgia pain, ilioinguinal neuralgia, anterior abdominal wall entrapment syndrome, lateral femerocutaneous neuralgia, cancer associated pain, somatic pain, or visceral pain. In some embodiments, the pain is opioid withdrawal pain. In some embodiments, the pain excludes one or more of neuropathic pain, radicular pain associated with degenerative disc disease, diabetic neuropathy pain, complex regional pain syndrome, peripheral ischemic neuropathy pain, small fiber neuropathy pain, large fiber neuropathy pain, neuropathy associated with neurofibromatosis, post herpetic neuralgia pain, ilioinguinal neuralgia, anterior abdominal wall entrapment syndrome, lateral femerocutaneous neuralgia, cancer associated pain, somatic pain, and visceral pain.

C. Combination Therapy

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

Where a method of treating pain is employed, the disclosed compositions (e.g., demeclocycline, chlortetracycline, and/or minocycline) may be provided to an individual together with an additional therapeutic. An additional therapeutic may be a compound or agent capable of treating pain. In some embodiments, an additional therapeutic is an analgesic. An EphB receptor inhibitor of the present disclosure may be provided in combination with an analgesic, thereby improving the efficacy of the pain treatment compared with the analgesic alone. An analgesic may be, for example, an opioid, a nonsteroidal anti-inflammatory drug (NSAID), acetaminophen, a steroid, a COX-2 inhibitor, or a topical analgesic (e.g., lidocaine, capsaicin, menthol, etc.). Any combination of two or more analgesics may be provided in comination with an EphB receptor inhibitor of the present disclosure.

An additional therapeutic may be a compound or agent capable of treating a neurological condition. For example, an EphB receptor inhibitor of the present disclosure may be provided in combination with an anticonvulsant (e.g., a gabapentinoid) and/or an antidepressant (e.g., a selective serotonin reuptake inhibitor), thereby simultaneously and/or sequentially treating multiple symptoms of a neurological condition.

Various combination regimens of the therapeutic agents and treatments may be employed. Non-limiting examples of such combinations are shown below, wherein a therapeutic agent such as a composition disclosed herein (e.g., a composition comprising demeclocycline, chlortetracycline, and/or minocycline) is “A” and a second agent, such as an additional therapeutic (e.g., analgesic, anticonvulsant, antidepressant, etc.) described herein or known in the art is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

In some embodiments, more than one course of therapy may be employed. It is contemplated that multiple courses may be implemented.

### V. Kits

Certain aspects of the disclosure also encompass kits for performing the methods of the disclosure, such as treatment of EphB receptor-associated conditions (e.g., pain). Embodiments relate to kits comprising the therapeutic pharmaceutical compositions of the disclosure. The kits may be useful in the treatment methods of the disclosure.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific therapeutic or non-therapeutic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

In some embodiments, when the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

### VI. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. The Examples should not be construed as limiting in any way. The contents of all cited references (including literature references, issued patents, published patent applications, and GenBank Accession numbers as cited throughout this application) are hereby expressly incorporated by reference. When definitions of terms in documents that are incorporated by reference herein conflict with those used herein, the definitions used herein govern.

### Example 1—Virtual Screening and Docking

A. Material and Methods

FDA Approved Small Molecules Preparation. The U.S. Food and Drug Administration approved drug database (2037 small molecules) was downloaded (available on the World Wide Web at drugbank.ca) and three dimensional (3D) structures were energy minimized using MMFF94 force field.

X-ray Crystal Structures Preparation. Crystal structure of the EphB1 catalytic domain has been resolved in the Protein data bank (PDB IDs: 3ZFX and 5MJA). Staurosporine, an EphB4 kinase inhibitor, was used as a query for ligand based drug design due to the scaffold complexity that could offer potential diverse scaffolds. Due to their high degree of amino acid sequence identity, the residues of the EphB1 catalytic domain have been previously assigned for docking based on EphB4 in case of (PDB ID: 3ZFX) along with staurosporine, showing high degree of sequence alignment similarity (87%). Other X-ray crystal structures were used for EphB2, EphB3, and EphB4 can be accessed at PDB IDs: 3ZFM, 3ZFY, and 3ZEW, respectively.

Ligand Based In Silico Screening. The energy minimized three-dimensional structure comparison between staurosporine and the FDA approved small molecules library was performed by Molecular Operating Environment (MOE) to assign the degree of structural similarity. The top scoring compounds were selected based on Tanimoto co-efficient score ranging from 1-0.5 to undergo docking.

Structure Based in silico Screening and scoring. The top ten selected energy minimized approved small molecules, based on literature survey and clinical indications, underwent docking simulations using MOE along with EphB1 kinase domain (PDB ID; 5MJA). The top selected energy minimized compounds underwent protonation state to add the missing hydrogens for proper ionization states (24-26). MOE Dock application was used to find the favorable binding conformations of the studied ligands. This was followed by scoring and assessment using the London dG Scoring method to show the potential hydrophobic and hydrogen bond interactions. The figures were generated by Chiemra (27), while the 2D generation was done using MOE tools.

B. Results

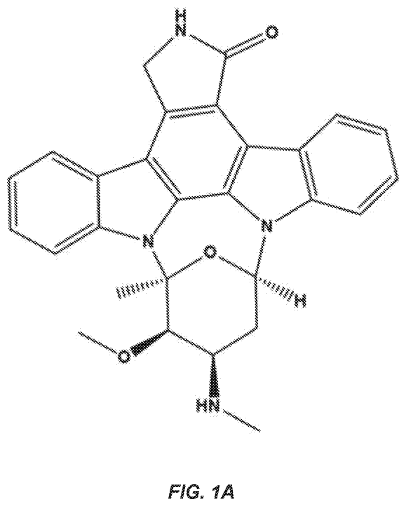

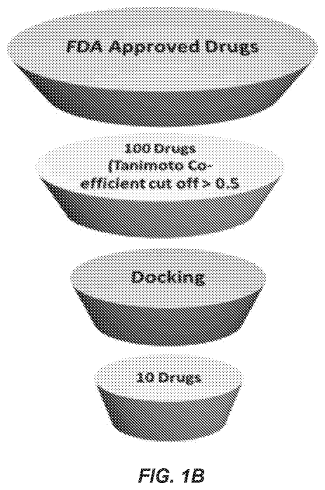

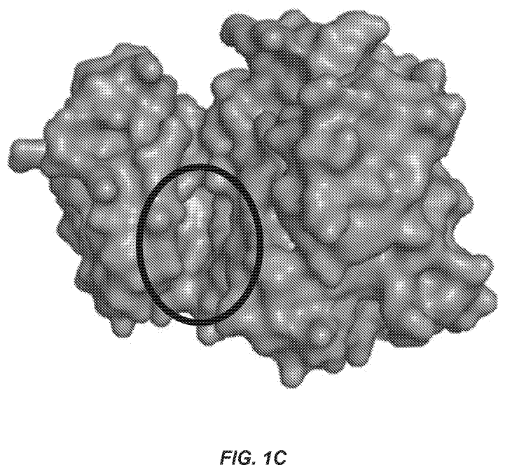

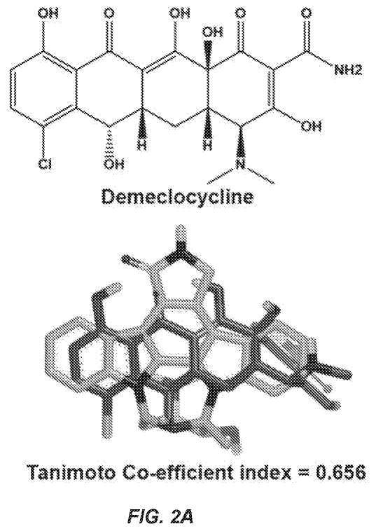

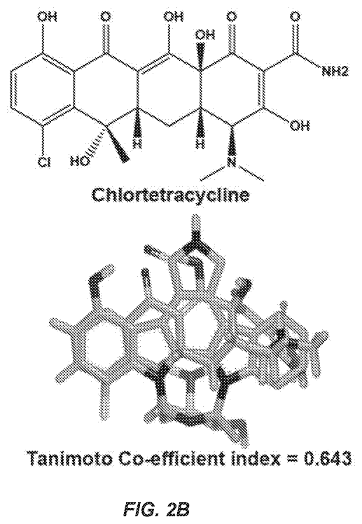

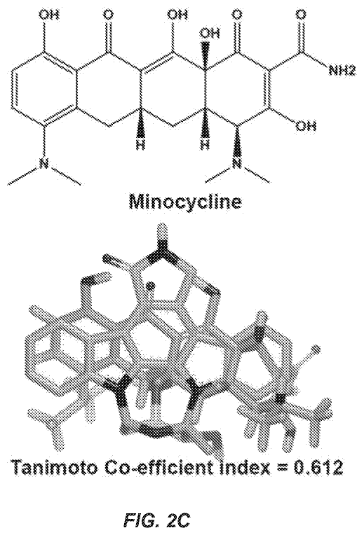

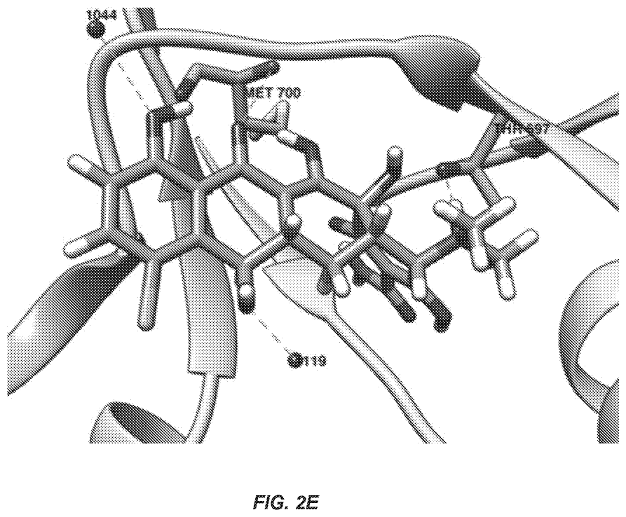

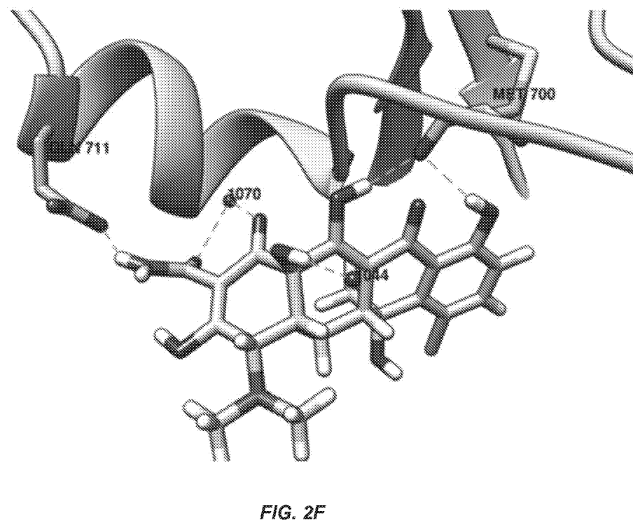

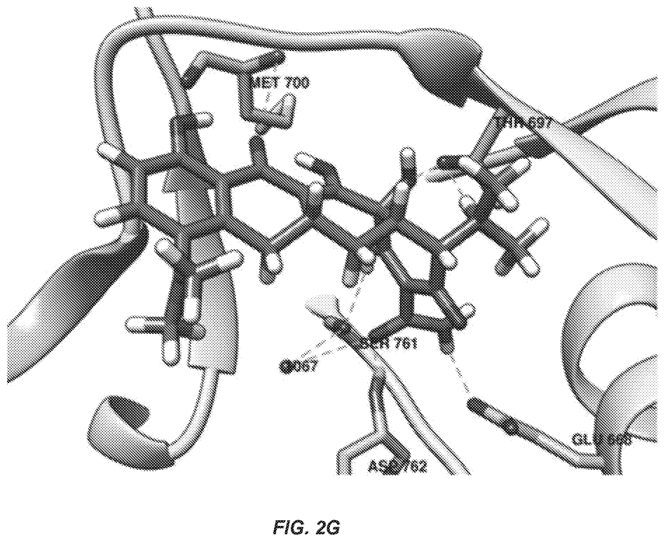

EphB kinases have been reported to have highly conserved ATP catalytic binding domains (28-29). Different crystal structures for EphB kinases have been resolved and can be accessed in the protein data bank (PDB); such as 3ZFX and 5MJA (EphB1), 3ZFM (EphB2), 3ZFY (EphB3), and 3ZEW (EphB4). Herein, the inventors studied staurosporine co-crystallized with EphB4 as a starting query for screening the MMFF94 energy-minimized library of FDA approved small molecules (2037 drugs). library underwent rapid overlay chemical structures to detect the molecular similarity based on staurosporine (FIG. 1A) using Tanimoto Co-efficient scores with cut off value 0.5. The top 100 drugs were further filtered on the basis of literature survey and exclusion of drugs with undesirable clinical indications/side effects, followed by in silico docking study along with catalytic binding domain of EphB1 protein kinase (PDB ID: 5MJA) ending up with 10 drugs, as shown in FIGS. 1B and 1C. Demeclocycline, chlortetracycline, rolitetracycline, oxytetracycline, ertapenem, darifenacin, quinagolide, minocycline, ramelteon, and galantamine showed proper binding affinity with respect to energy scoring, Tanimoto scores, and binding mode towards EphB1 kinase domain suggesting hydrophobic—hydrophobic and network of hydrogen bonds interactions (FIGS. 2A-2G)

### Example 2—In Vitro Biological Evaluation

A. Material and Methods

1. EphB1 Protein Kinase IC₅₀ Profiling.

Seven FDA approved drugs were selected to enroll a radiometric protein kinase assay (33P PanQinase® Activity Assay, ProQinase) to measure the effect of increasing concentrations of compound on catalytic activity of the EphB1, EphB2, EphB3, and EphB4 kinase domains. Kinase domains were produced by ProQinase using human cDNAs to express recombinant GST/His-fusion proteins that were purified by affinity chromatography and determined to be enzymatically active by phosphorylation of a Poly (Glu, Tyr) substrate. The FDA compounds were assayed in 10 concentrations in the range from 1×10⁻⁴ M to 3×10⁻⁹ M for their ability to effect kinase activities. The final DMSO concentration in the reaction cocktails was 1% in all cases. Kinase assays were performed in 96-well FlashPlates™ from PerkinElmer (Boston, Mass., USA) in a 50 μl reaction volume. The reaction cocktail was pipetted in four steps in the following order: 20 μl of assay buffer (standard buffer), 5 μl of ATP solution (in H₂O), 5 μl of test compound (in 10% DMSO), and 20 μl enzyme/substrate mix. The assay for the protein kinase contained 70 mM HEPES-NaOH pH 7.5, 3 mM MgCl₂, 3 mM MnCl₂, 3 μM Na-orthovanadate, 1.2 mM DTT, 50 μg/ml PEG20000, ATP (corresponding to the apparent ATP-Km of the kinase), [γ-³³P]-ATP (approx. 2×10⁵ cpm per well), protein kinase, and substrate. The reaction cocktails were incubated at 30° C. for 60 minutes. The reaction was stopped with 50 μl of 2% (v/v) H₃PO₄, plates were aspirated and washed two times with 200 μl 0.9% (w/v) NaCl. Incorporation of ³³Pi was determined with a microplate scintillation counter (Microbeta, Wallac). All assays were performed with a BeckmanCoulter/SAGIAN™ Core System. The median value of the counts in column 1 (n=8) of each assay plate was defined as “low control”. This value reflects unspecific binding of radioactivity to the plate in the absence of a protein kinase but in the presence of the substrate. The median value of the counts in column 7 of each assay plate (n=8) was taken as the “high control”, i.e. full activity in the absence of any inhibitor. The difference between high and low control was taken as 100% activity. As part of the data evaluation the low control value from a particular plate was subtracted from the high control value as well as from all 80 “compound values” of the corresponding plate. The residual activity (in %) for each well of a particular plate was calculated by using the following formula: Res. Activity (%)=100×[(cpm of compound−low control)/(high control−low control)]

The residual activities for each concentration and the compound IC₅₀ values were calculated using Quattro Workflow V3.1.1 (Quattro Research GmbH, Munich, Germany; available on the World Wide Web at quattro-research.com). The fitting model for the IC₅₀ determinations was “Sigmoidal response (variable slope)” with parameters “top” fixed at 100% and “bottom” at 0%. The fitting method used was a least-squares fit.

2. Differential IC₅₀ Profiling for EphB Protein Kinase Family.

Chloride salts of demeclocycline, chlortetracycline, minocycline, dual Equimolar combination of demeclocycline/minocycline (DM), demeclocycline/chlortetracycline (DC), minocycline/chlortetracycline (MC), and triple Equimolar combination of minocycline/chlortetracycline/demeclocycline (MCD) were selected to be screened against EphB1, EphB2, EphB3, and EphB4 and using 33PanQinase® Activity Assay, as previously described. For each kinase, the median value of the cpm of three wells with complete reaction cocktails, but without kinase, was defined as “low control” (n=3). This value reflects unspecific binding of radioactivity to the plate in the absence of protein kinase but in the presence of the substrate. Additionally, for each kinase the median value of the cpm of three other wells with the complete reaction cocktail, but without any compound, was taken as the “high control”, i.e. full activity in the absence of any inhibitor (n=3). The difference between high and low control was taken as 100% activity for each kinase. As part of the data evaluation the low control of each row of a particular plate was subtracted from the high control value as well as from their corresponding “compound values”. The residual activity (in %) for each well of each row of a particular plate was calculated by using the following formula: Res. Activity (%)=100×[(cpm of compound−low control)/(high control−low control)]. Since 10 distinct concentrations of each test compound were tested against each kinase, the evaluation of the raw data resulted in 10 values for residual activities per kinase. Based on each 10 corresponding residual activities, IC₅₀ values were calculated using Prism 5.04 for Windows (Graphpad, San Diego, Calif., USA). The mathematical model used was “Sigmoidal response (variable slope)” with parameters “top” fixed at 100% and “bottom” at 0%.

B. Results

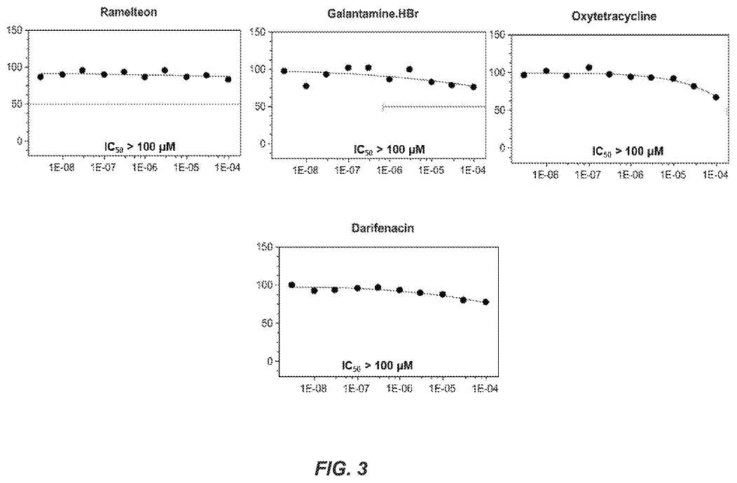

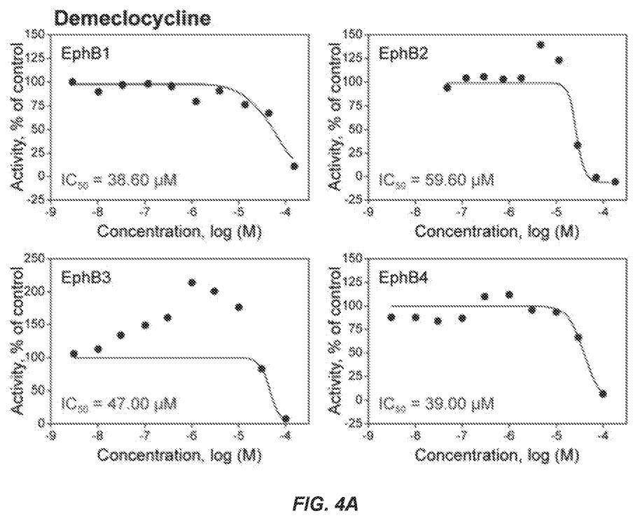

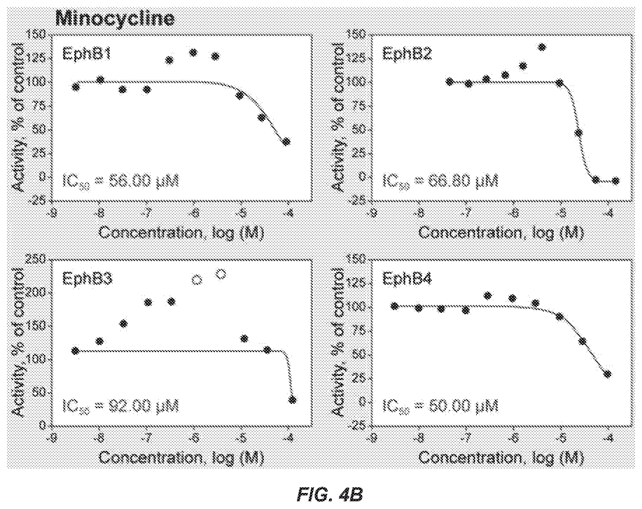

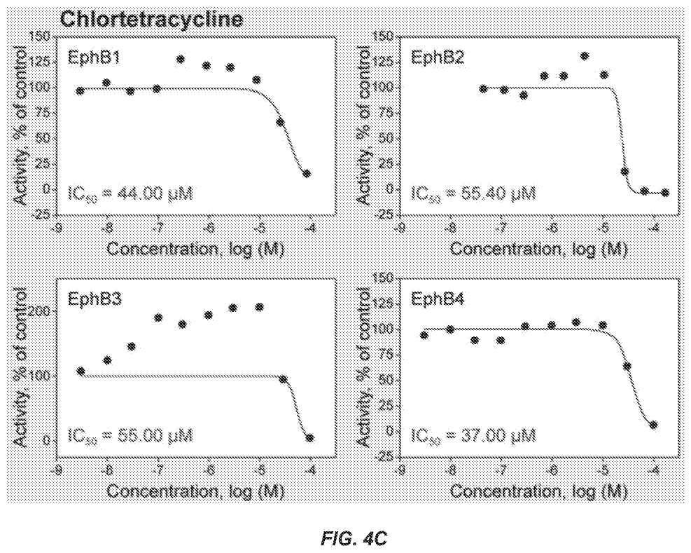

Seven of the identified FDA approved drugs were first tested for their inhibitory profiles in an in vitro EphB1 protein kinase assay. Ramelteon, Oxytetracycline, Galantamine, and Darifenacin failed to inhibit EphB1 kinase activity at concentrations up to 100 μM (FIG. 3). However, demeclocycline, chlortetracycline, and minocycline inhibited the EphB1 kinase domain with IC₅₀ calculated to be 39, 44, and 56 μM, respectively (FIGS. 4A-4C).

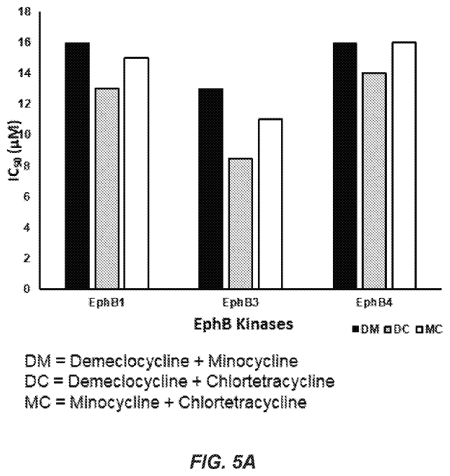

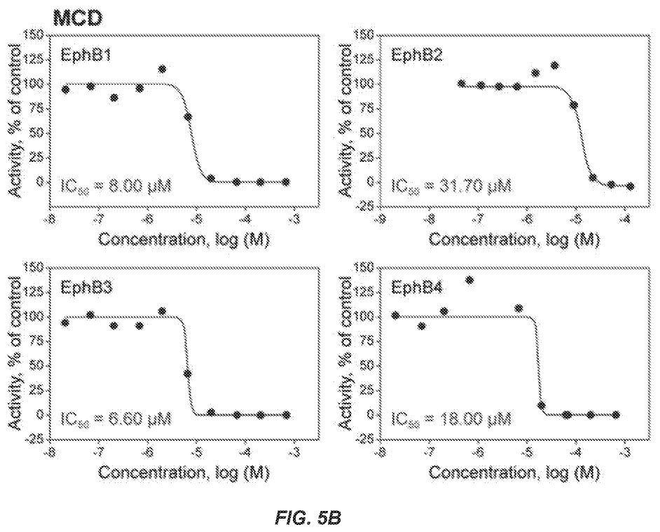

This suggested the potential of tetracycline skeleton to be ATP competitive inhibitors within the kinase binding domain. This prompted the inventors to further screen demeclocycline, chlortetracycline, and minocycline against EphB2, EphB3 and EphB4 protein kinases, revealing they too were inhibited with IC₅₀ ranging from 37-92 μM (FIGS. 4A-4C). Demeclocycline, chlortetracycline, and minocycline all showed a similar biphasic influence on the EphB3 catalytic activity, in the low nanomolar ranges they slightly elevated activity, whereas at higher concentrations they inhibited activity with IC₅₀ of 47, 55, and 92 μM, respectively. Such biphasic responses were not observed with the EphB1, EphB2, and EphB4 kinase domains. The IC₅₀ against EphB kinases for two-drug and three-drug combinations was investigated for demeclocycline (D), chlorotetracycline (C), and minocycline (M). Equimolar ratios of DC (demeclocycline+chlorotetracycline), DM (demeclocycline+minocycline), and MC (minocycline+chlorotetracycline) were used and showed improvement in the IC₅₀ against EphB1 at 16, 13, and 15 μM, respectively. Exploring the triple drug strategy by having equimolar ratio of MCD improved the IC₅₀ up to 8 μM. (FIGS. 5A and 5B).

### Example 3—In Vivo Biological Evaluation

A. Material and Methods

Animals. A total of 40 male outbred CD1 mice were obtained from Charles River Laboratories at 8 weeks of age. Mice were housed in the animal facility of UT southwestern Medical Center, with constant temperature (21-24° C.) and humidity (30-50%) with free access to standard animal feed and water. The room was kept on a 12/12 light/dark cycle, with white light (light cycle) on at 2400 hours and red lights (dark cycle) on at 1200 hours. All of the procedures were conducted with approval from the UT southwestern Medical Center Institutional Animal Care and Use Committee (IACUC Protocol No. 2017-102090). Ugo Basile® the original Plantar Test (Hargreaves Apparatus) was used for thermal stimulation, where infrared beam was adjusted to give an average paw withdrawal latency of about 10 s in wild type mice and cut-off time was set to 30 s to avoid tissue damage. Ugo Basile® Dynamic Plantar Aesthesiometer (DPA) for mechanical stimulation (Electronic Von Frey) were used for mechanical stimulation, where the force (grams) was set at 50 g to prevent tissue damage. Animals were placed in clear acrylic cubicles (22×16.5×14 cm) for at least one hour prior testing. The testing/recording was done every 5 minutes with no repetition for the same mouse. Blinded person(s) to the individual treatment assignments recorded true reflexes.

Drugs. Demeclocycline, minocycline, and chlorotetracycline were freely soluble in phosphate buffer saline (PBS) at their dose level, purchased from Sigma Aldrich. DC was prepared by having equimolar ratio of demeclocycline and chlorotetracycline dissolved in PBS; 100 μl of the whole solution was administered per mouse via oral gavage. MCD was prepared by having equimolar ratio of demeclocycline, minocycline, and chlorotetracycline dissolved in PBS; 100 μl of the whole solution was administered per mouse via oral gavage. Capsaicin (dissolved in 5% Tween 80 and 5% ethanol and brought to volume with PBS), where five μl was injected in the right paw of the mice. Complete freund's adjuvant (CFA, contained 1 mg of Mycobacterium tuberculosis (H37Ra, American Type Culture Collection 25177) per milliliter of emulsion in 85% paraffin oil and 15% mannide monooleate), where 5 μl was injected in the right paw of the mice.

Capsaicin Induced pain model. Four different groups of CD-1 WT mice (n=5, each) were assigned to represent four different treatments including; control (PBS), Demeclocycline (20 mg/kg/day/p.o), DC (7 mg/kg/day/p.o), and MCD (5 mg/kg/day/p.o). The drugs were administered via gavage three consecutive days before capsaicin injection. Capsaicin was injected in the right paw, while the left paw remained un-injected as control. The testing involved two stimulus thermal hyperalgesia using Hargreaves Test and mechanical allodynia using Von Frey Test. The whole experiment was conducted within 1-2 hours.

Complete freund's adjuvant (CFA) Induced pain model. Four different groups of CD-1 WT mice (n=5, each) were assigned to represent four different treatments including; control (PBS), Demeclocycline (20 mg/kg/day/p.o), DC (7 mg/kg/day/p.o), and MCD (5 mg/kg/day/p.o). A baseline was for detected for the mice behavior towards thermal hyperalgesia using Hargreaves Test and mechanical allodynia using Von Frey Test. The drugs were administered via gavage for three consecutive days before CFA injection. On the morning of the experiment (6 hours before CFA injection), the last dose of drugs was administered. CFA was injected in the right paw, while the left paw remained un-injected as control. The testing involved two stimulus thermal hyperalgesia using Hargreaves Test and mechanical allodynia using Von Frey Test at Days 1, 2, 3, and 5.

Brain lysate and western blotting. Brains were isolated and lysed in RIPA buffer with the addition of Complete protease inhibitor cocktail (Roche). Protein concentration was quantified using Pierce BCA protein assay kit (Pierce Biotechnology), with three biological replicates. After separation via SDS-PAGE, proteins were transferred to nitrocellulose membranes (Bio-Rad), blocked in 5% skim milk/TBS and incubated with appropriate primary antibodies as following: anti-Eph receptor B1+Eph receptor B2 (phospho Y594+Y604) antibody (Abcam, ab61791, 1:200), anti-Human/Mouse EphB2 Antibody (R&D system, AF467, 1:1000), anti-Gapdh (Sigma, AB2302, 1:5000). Horseradish peroxidase (HRP)-conjugated peroxidase anti-rabbit, anti-chicken, or anti-goat antibodies (1:25000-1:50000) were used as secondary antibodies. The membranes were explored using Licor Odyssey Fc system and quantified by Image Studio software.

Spinal cord Isolation. The spinal cords were isolated from the spinal column by hydraulic extrusion method 60. The spinal cords were fixed in 4% paraformaldehyde for 2 hr at 4 C°. After washing with PBS, the spinal cords were placed in 30% sucrose overnight at 4 C° and embedded in OCT compound for Cryo section, with three biological replicates.

Immunostaining. Prior to the immunostaining, spinal cord sections were post fixed in 4% paraformaldehyde in PBS for 10 minutes at room temperature and washed with PBS. For antigen retrieval, the sections were steamed in epitope retrieval solution (IHC World) for 20 minutes, and cool down for 30 minutes at room temperature. After washing with PBS, the sections were incubated in 10% normal goat serum with 0.3% Triton X for 30 minutes. Primary anti-bodies were incubated overnight at 4° C. The sections were subsequently washed with PBS and incubated with corresponding secondary antibodies conjugated to Alexa Fluor 488 or 555 (Invitrogen). DAPI stained for 5 minutes. The sections were mounted in anti-fade mounting medium (Vector Laboratories, Burlingame, Calif.). Primary antibodies used were anti-NeuN (Abcam, ab104224, 1:100) and anti-pEphB 1/2 (Abcam, ab61791, 1:100; Eph receptor B1+Eph receptor B2 (Y594+Y604)).

B. Results

1. Capsaicin Induced Pain Model

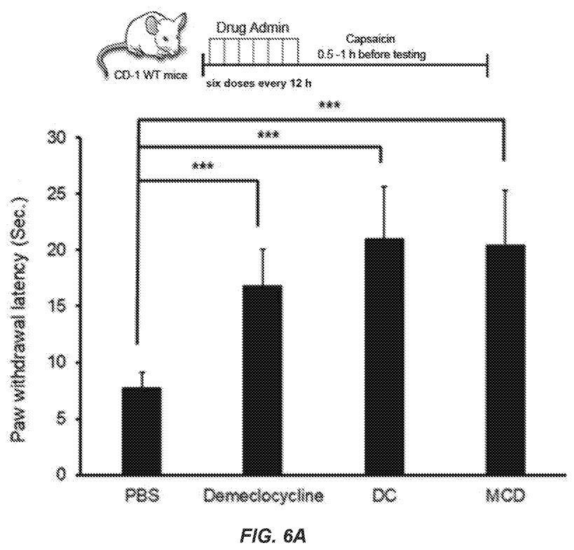

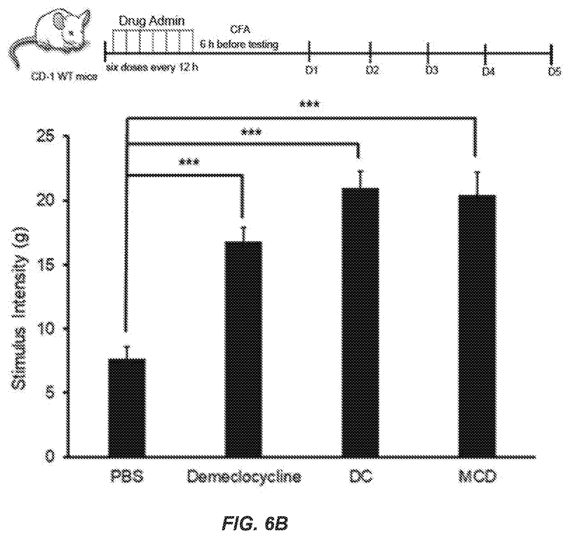

Demeclocycline (17 mg/kg/day, p.o), DC (7 mg/kg/day, p.o), and MCD (5 mg/kg/day, p.o) prolonged the paw withdrawal latency of the injected paw with capsaicin significantly compared to the controls upon exposure to thermal stimulus. The same behavior was observed upon exposure to mechanical stimulus with the three tested formulas, as shown in FIGS. 6A and 6B.

2. Complete Freund's Adjuvant (CFA) Induced Pain Model

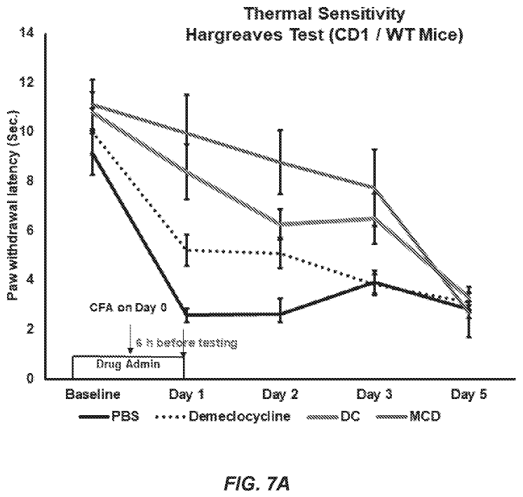

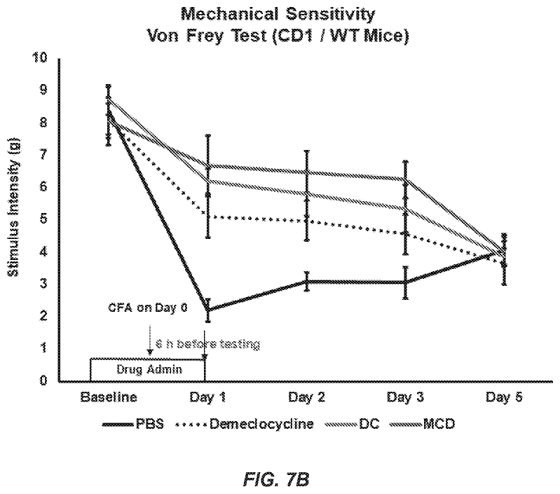

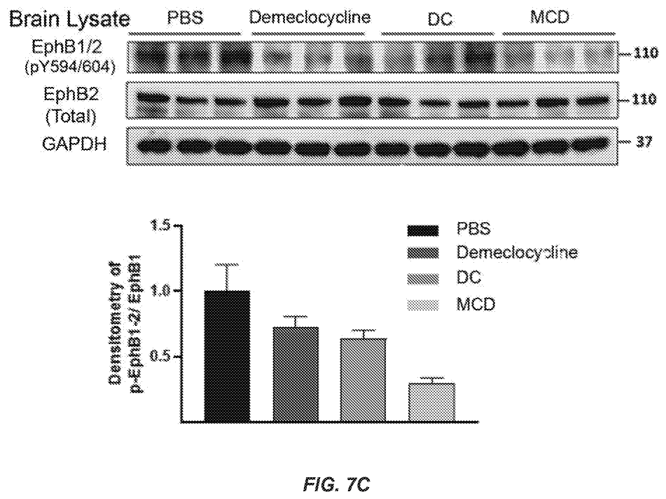

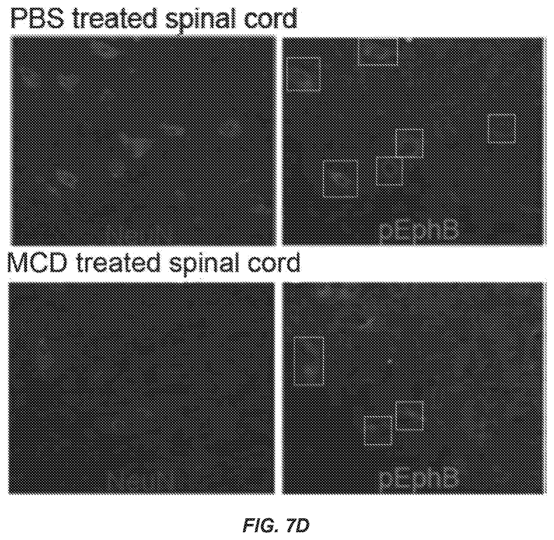

Demeclocycline, DC, and MCD prolonged the paw withdrawal latency of the injected paw with capsaicin significantly compared to the controls upon exposure to thermal stimulus. The same behavior was observed upon exposure to mechanical stimulus with the three tested formulas, as shown in FIGS. 7A and 7B. Demeclocycline showed significant improvement in the paw withdrawal latency for thermal hyperalgesia stimulus between time points until Day 2 (F (2.451, 34.317)=55.960, ***P<0.001), however it only showed significant improvement in reversing the tactile allodynia stimuli at day 1 (F (3.441, 44.737)=28.999, ***P<0.001); suggesting that the demeclocycline effect was diminished by Day 2 significantly. DC showed significant improvement in the paw withdrawal latency for thermal hyperalgesia and mechanical allodynia stimuli between time points until Day 3 (F (2.072, 29.012)=31.885, ***P<0.001; and F (2.125, 19.122)=24.320, ***P<0.001), respectively; suggesting that the DC effect was diminished by Day 5 significantly. MCD showed significant improvement in the paw withdrawal latency for thermal hyperalgesia and mechanical allodynia stimuli between time points until Day 3 (F (2.015, 28.215)=16.419, ***P<0.001; and F (2.471, 24.711)=14.525, ***P<0.001), respectively; suggesting that the MCD effect was diminished by Day 5 significantly.

Brain tissue was isolated from mice treated with PBS, demeclocycline, DC, or MCD to extract protein lysate and monitor the pEphB 1/2 levels by western blot. MCD treated samples significantly inhibited the phosphorylation of EphB 1/2 (FIG. 7C). In addition, spinal cord was isolated from mice treated with PBS and MCD after fixing for immunostaining along with NeuN (neurons) and pEphB 1/2, the activated form of EphB. pEphB 1/2 signal was inhibited in the samples treated with MCD compared to PBS treated samples, indicating that MCD inhibits the phosphorylation of EphB 1/2 (FIG. 7D).

### Example 4—Analysis of Structural Basis for EphB1 Inhibition

A. Material and Methods

1. Protein Expression and Purification

hEphb1 with residues 602-896 was subcloned into pETDuet vector with non-cleavable N terminal 6×His tag and transformed into Rosetta (DE3) pLysS cells (Novagen). Target protein was expressed in cultures grown in autoinduction media at 18° C. overnight (30). The culture was harvested and sonicated in lysis buffer (50 mM Tris (pH8.0), 1M NaCl, 1 mM DTT and supplemented with protease inhibitors). The lysate was centrifuged, the supernatant was loaded onto a Ni-NTA affinity column (Qiagen) and the beads were washed with wash buffer (20 mM Tris (pH8.0), 1 M NaCl, 1 mM DTT and 20 mM Imidazole (pH 8.0)) and eluted with elution buffer (20 mM Tris (pH 8.0), 150 mM NaCl, 1 mM DTT and 250 mM Imidazole (pH 8.0)). The eluate was concentrated and further purified by gel filtration chromatography. The peak fractions were collected and concentrated to about 10 mg/mL for crystallization screening. 2. Crystallization and structure determination

The crystals of the apo hEphb1 were obtained using the hanging-drop, vapor-diffusion method by mixing 1 μL protein (10 mg/mL) with 1 μL reservoir solution containing 0.2M Sodium Malonate (pH4.6) and 14% PEG 3350 and incubating at 18° C. The crystals were observed after two days and reached the maximum size after five days. The complex crystals with chlortetracycline were generated by soaking the apo crystals with 30 mM compound for 6 hours at 18° C. The datasets were collected at APS-19-ID at wavelengths of 0.97926 Å. Data was indexed, integrated and scaled by the program HKL3000 (31). Phases were determined by molecular replacement using the apo Ephb1 structure (PDB code: 3ZFX) as a searching model. The model was further built manually with COOT (32) and iteratively refined using Phenix.refine (33). The PROCHECK program was used to check the quality of the final model, which shows good stereochemistry according to the Ramachandran plot (34). All structure figures were generated by using the PyMOL Molecular Graphics System, Schrödinger, LLC. Software used in this work was curated by SBGrid (35).

B. Results

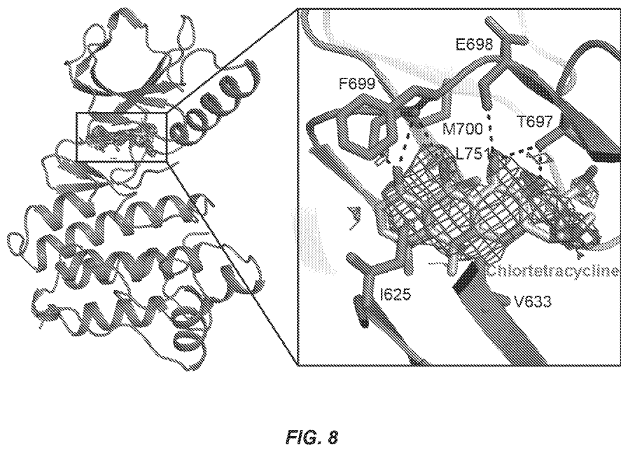

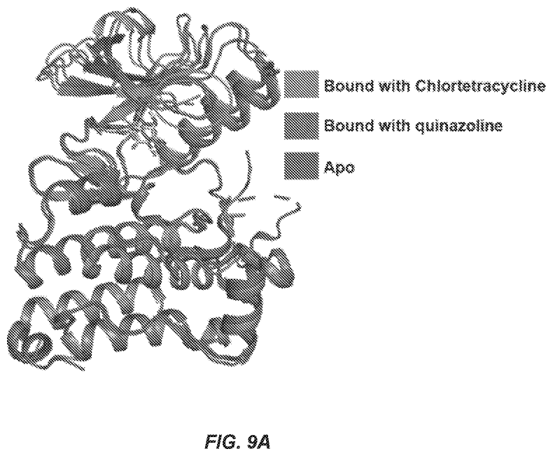

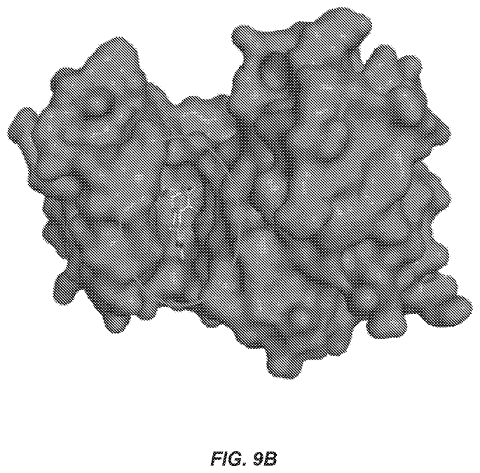

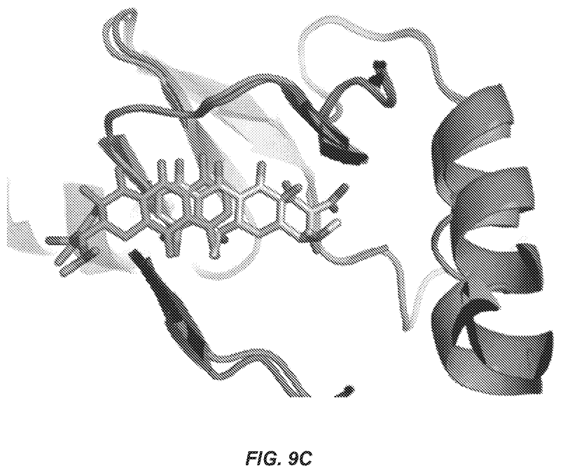

To investigate the structural basis of the tetracycline skeleton in EphB1 inhibition, the crystal structure of EphB1 in complex with chlortetracycline was determined by soaking the crystals in the solution containing the compound. FIG. 8 shows a cartoon representation of the overall structure of EphB1 bound to chlortetracycline. The composite omit map contoured at 1.0 shows clear density (mesh) for chlortetracycline, shown in stick representation. The overall structure of EphB1 bound with chlortetracycline adopts the traditional bi-lobed kinase fold similar to the structure of EphB1 bound to a quinazoline-based inhibitor (PDB code: 5MJA) with core root mean-square deviation (RMSD) of 0.4 Å for 197 Ca atoms. FIG. 9A shows a comparison of the structures of EphB1 bound to chlortetracycline, EphB1 bound to quinazoline, and apo EphB1. The inhibitor bound structure of EphB1 adopts a more closed conformation than apo EphB1 structure, with the glycine rich loop folding tightly over the ligand. For the chlortetracycline bound structure, clear electron density for chlortetracycline is visible near the catalytic pocket (FIG. 8) which is similar to the quinazoline-based inhibitor binding pocket (FIG. 9B). Chlortetracycline is stabilized in the catalytic pocket by numerous favorable interactions (FIG. 8). The first three aromatic rings are sandwiched by 1625, V633, F699 and L751 and there are several hydrogen bond interactions with the side chain of T697 and the main chain of E698 and M700, which is also consistent with the prediction (FIG. 9C).

### Example 5—MCD in an Orthotopic Glioma Tumor Model

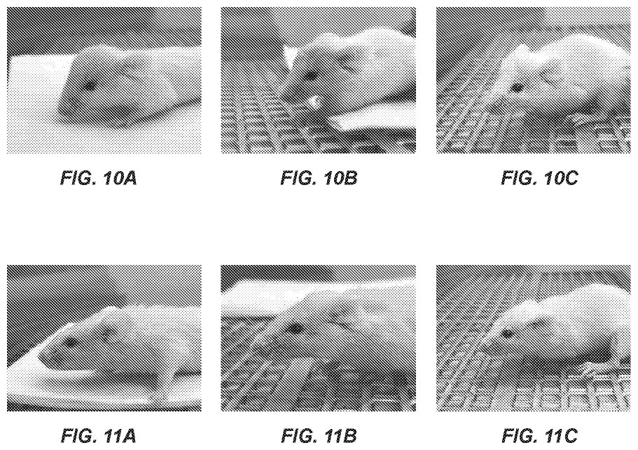

In a first set of studies, primary mouse glioma cells derived from the derived from the triple-mutant glioma (Glast-CreERT²×p53^{f/f}, PTEN^{f/f}, and _{LSL}BRAFV^{600E}) model were injected (250K cells) into the ventral mouse forebrain (caudate) at precisely the same exact coordinates of 6 week old NSG immunodeficient mice. Seven days following injection, mice were split into a treatment group and a vehicle group. Treatment group mice were given a combination of minocycline, chlortetracycline, and demeclocycline (MCD) formulated in PBS at 20 mg/kg B.I.D by oral gavage for five weeks, compared with no treatment for the vehicle group. Animal health was monitored daily. FIGS. 10A-11C show example images of untreated mice harboring glioma tumors. FIGS. 11A-11C show example images of mice treated with MCD, showing that mice treated with MCD have significantly reduced tumor size compared with untreated mice.

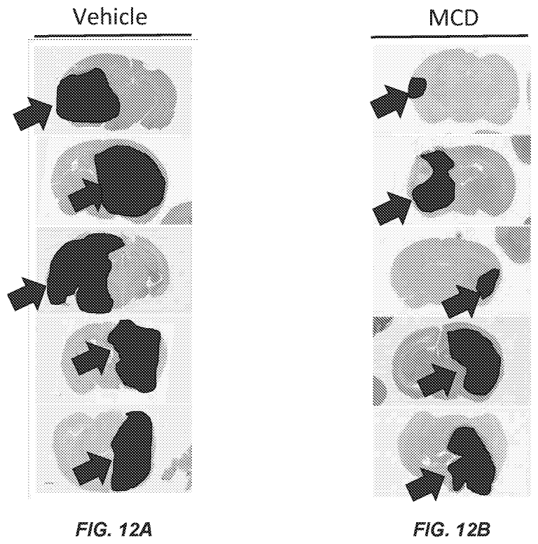

In a second set of studies, mouse glioma cells derived from the derived from the triple-mutant glioma (Glast-CreERT²×p53^{f/f}, PTEN^{flf}, and _{LSL}BRAFV^{600E}) model were injected (250K cells) into the ventral mouse forebrain (caudate) at precisely the same exact coordinates of 6 week old NSG immunodeficient mice. Seven days following injection, mice were split into a treatment group and a vehicle group. Treatment group mice were given a combination of minocycline, chlortetracycline, and demeclocycline (MCD) formulated in PBS at 20 mg/kg B.I.D by oral gavage for 15 days, compared with no treatment for the vehicle group. Animal health was monitored daily. FIGS. 12A and 12B show representative H&E stained histological brain images, demonstrating that MCD treatment reduces tumor size in mouse brain tissue. Dark areas show tumor area.

### Example 6—Additional Analysis of an Orthotopic Glioma Tumor Model

NSG mice are injected with triple-mutant glioma cells and treated as described in Example 5. Animal health is monitored daily and mice are weighed until clinical thresholds are reached that require sacrifice. Overall duration (days) of survival are used to perform Kaplan-Meier survival analysis. At the time of sacrifice, mice are deeply anesthetized with Avertin and cardiac perfused with PBS followed by 4% paraformaldehyde (PFA). Whole brains are cut into 1 mm coronal sections and stained using H&E staining and subjected to immunohistochemical analysis. Histology images are scanned for digital reconstruction of tumor volume and quantification of correlative markers.

### Example 7—MCD in a Basic Glioma Tumor Model

Triple-mutant glioma (Glast-CreERT2×p53f/f, PTENflf, and LSLBRAFV^{600E}) mice are given tamoxifen at 1 mg/kg intraperitoneally five times. Following this, mice are split into a treatment group and a vehicle group. Treatment group mice are given a combination of minocycline, chlortetracycline, and demeclocycline (MCD) formulated in PBS at 20 mg/kg B.I.D by oral gavage for five weeks, compared with no treatment for the vehicle group. Animal health is monitored daily, and mice are weighed until clinical thresholds are reached that require sacrifice. Sacrifice is performed using inhalation overdose of CO₂. Overall duration (days) of survival are used to perform Kaplan-Meier survival analysis. At the time of sacrifice, mice are deeply anesthetized with Avertin and cardiac perfused with PBS followed by 4% paraformaldehyde (PFA). Whole brains are cut into 1 mm coronal sections and stained using H&E staining and subjected to immunohistochemical analysis.

### Example 8—In Vitro Analysis of Demeclocycline and MCD Treatment

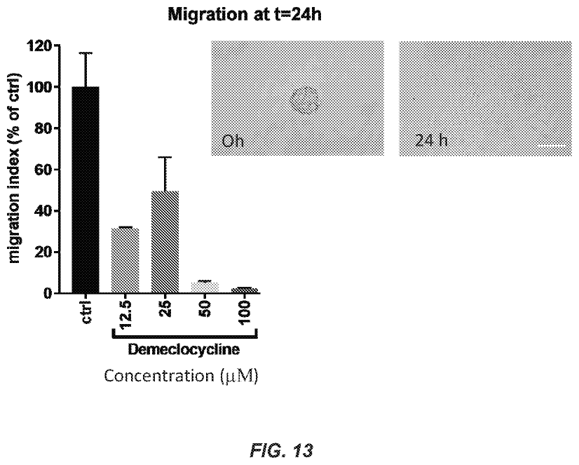

Human glioblastoma (GBM) cells were cultured with demeclocycline at concentrations of 0 (ctrl), 12.5, 25, 50, and 100 μM. Cell migration was tested after 24 hours. FIG. 13 shows that demeclocycline inhibited glioblastoma cell migration at all of the concentrations tested.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references and the publications referred to throughout the specification, to the extent that they provide exemplary procedural or other details supplementary to those set forth herein, are specifically incorporated herein by reference.
1. Pasquale E B. Eph receptors and ephrins in cancer: bidirectional signaling and beyond. Nature reviews Cancer. 2010; 10(3):165-180.
2. Xi H-Q, Wu X-S, Wei B, Chen L. Eph receptors and ephrins as targets for cancer therapy. Journal of Cellular and Molecular Medicine. 2012; 16(12):2894-2909. doi:10.1111/j.1582-4934.2012.01612.x.
3. Day B W, Stringer B W, Boyd A W. Eph receptors as therapeutic targets in glioblastoma. British Journal of Cancer. 2014; 111(7):1255-1261. doi:10.1038/bjc.2014.73.
4. O'Neal W T, Griffin W F, Dries-Devlin J L, et al. Ephrin-Eph signaling as a potential therapeutic target for the treatment of myocardial infarction. Medical hypotheses. 2013; 80(6):10.1016/j.mehy.2013.02.024. doi:10.1016/j.mehy.2013.02.024.
5. Overman R, Debreczeni J, Truman C, McAlister M, Attwood T. Completing the structural family portrait of the human EphB tyrosine kinase domains. Protein Sci. 2014; 23(5):627-38.
6. Cowan, C. A. and Henkemeyer, M. Ephrins in reverse, park, and drive. Trends in Cell Biology, 2002; 12: 339-346.
7. Dai D, Huang Q, Nussinov R, Ma B. Promiscuous and specific recognition among Ephrins and Eph receptors. Biochimica et biophysica acta. 2014; 1844(10):1729-1740. Noberini R, Lamberto I, Pasquale E B. Targeting Eph Receptors with Peptides and Small Molecules: Progress and Challenges. Seminars in cell & developmental biology. 2012; 23(1):51-57.
8. Riedl S J, Pasquale E B. Targeting the Eph System with Peptides and Peptide Conjugates. Current drug targets. 2015; 16(10):1031-1047.
9. Fry D C, Vassilev L T. Targeting protein-protein interactions for cancer therapy. J Mol Med. 2005; 83:955-63.
10. Kung A, Chen Y, Schimpl M, Ni F, Zhu J, Turner M, Molina H, Overman R, Zhang C. Development of Specific, Irreversible Inhibitors for a Receptor Tyrosine Kinase EphB3. J. Am Chem Soc. 2016 Aug. 24; 138(33):10554-60.
11. Battaglia A A, Sehayek K, Grist J, McMahon S B, and Gavazzi I. (2003). EphB receptors and ephrin-B ligands regulate spinal sensory connectivity and modulate pain processing. Nat Neurosci 6: 339-40.
12. Kobayashi H, Kitamura T, Sekiguchi M, Homma M K, Kabuyama Y, Konno S, Kikuchi S, and Homma Y. (2007). Involvement of EphB1 receptor/EphrinB2 ligand in neuropathic pain. Spine 32: 1592-8.
13. Han Y, Song X S, Liu W T, Henkemeyer M, and Song X J. (2008). Targeted mutation of EphB1 receptor prevents development of neuropathic hyperalgesia and physical dependence on morphine in mice. Mol Pain 4: 60. PMCID: PMC2605438
14. Liu W T, Han Y, Li H C, Adams B, Zheng J H, Wu Y P, Henkemeyer M, and Song X J. (2009). An in vivo mouse model of long-term potentiation at synapses between primary afferent C-fibers and spinal dorsal horn neurons: essential role of EphB1 receptor. Mol Pain 5: 29. PMCID: PMC2704201
15. Song X J, Zheng J H, Cao J L, Liu W T, Song X S, and Huang Z J. (2008). EphrinB-EphB receptor signaling contributes to neuropathic pain by regulating neural excitability and spinal synaptic plasticity in rats. Pain 139: 168-80.
16. Song X J, Cao J L, Li H C, Zheng J H, Song X S, and Xiong L Z. (2008). Upregulation and redistribution of ephrinB and EphB receptor in dorsal root ganglion and spinal dorsal horn neurons after peripheral nerve injury and dorsal rhizotomy. Eur J Pain 12: 1031-9.
17. Liu S, Liu W T, Liu Y P, Dong H L, Henkemeyer M, Xiong L Z, and Song X J. (2011). Blocking EphB1 Receptor Forward Signaling in Spinal Cord Relieves Bone Cancer Pain and Rescues Analgesic Effect of Morphine Treatment in Rodents. Cancer Res 71: 4392-4402. PMID: 21555368
18. Dong Y, Mao-Ying Q L, Chen J W, Yang C J, Wang Y Q, and Tan Z M. (2011). Involvement of EphB1 receptor/ephrinB1 ligand in bone cancer pain. Neurosci Lett 496: 163-7.
19. Cibert-Goton V, Yuan G, Battaglia A, Fredriksson S, Henkemeyer M, Sears T, and Gavazzi I. (2013). Involvement of EphB1 receptors signalling in models of inflammatory and neuropathic pain. PLoS One 8: e53673. PMCID: PMC3547059
20. Slack S, Battaglia A, Cibert-Goton V, and Gavazzi I. (2008). EphrinB2 induces tyrosine phosphorylation of NR2B via Src-family kinases during inflammatory hyperalgesia. Neuroscience 156: 175-83.
21. Zhao J, Yuan G, Cendan C M, Nassar M A, Lagerstrom M C, Kullander K, Gavazzi I, and Wood J N. (2010). Nociceptor-expressed ephrin-B2 regulates inflammatory and neuropathic pain. Mol Pain 6: 77.
22. Liu W T, Li H C, Song X S, Huang Z J, and Song X J. (2009). EphB receptor signaling in mouse spinal cord contributes to physical dependence on morphine. FASEB J 23: 90-8.
23. Hanamura K, Washburn H R, Sheffler-Collins S I, Xia N L, Henderson N, Tillu D V, Hassler S, Spellman D S, Zhang G, Neubert T A, Price T J, and Dalva M B. (2017). Extracellular phosphorylation of a receptor tyrosine kinase controls synaptic localization of NMDA receptors and regulates pathological pain. PLoS Biol 15: e2002457.
24. Molecular Operating Environment (MOE), Chemical Computing Group Inc., 1010 Sherbooke St. West, Suite #910, Montreal, QC, Canada., H3A, 2R7. 2008.
25. Naim M, Bhat S, Rankin K N, et al. (2007) Solvated interaction energy (SIE) for scoring protein-ligand binding affinities. 1. Exploring the parameter space. J Chem Inf Model. 47(1):122-133.
26. Paul L. (2009) Protonate3D: assignment of ionization states and hydrogen coordinates to macromolecular structures. Proteins. 75: 187-205.
27. Pettersen E F, Goddard T D, Huang C C, Couch G S, Greenblatt D M, Meng E C, Ferrin T E. (2005) UCSF Chimera—a visualization system for exploratory research and analysis. J Comput. Chem. 25(13): 1605-12.
28. Himanen J-P, Henkemeyer M, and Nikolov D B. (1998). Crystal structure of the ligand-binding domain of the receptor tyrosine kinase EphB2. Nature 396: 486-491.
29. Himanen J P, Rajashankar K R, Lackmann M, Cowan C A, Henkemeyer M, and Nikolov D B. (2001). Crystal structure of an Eph receptor-ephrin complex. Nature 414: 933-8. PMID: 11780069
30. Studier, W. F. (2005). Protein production by auto-induction in high-density shaking cultures. Protein Expression and Purification 41, 207-234.
31. Minor, W., Cymborowski, M., Otwinowski, Z., and Chruszcz, M. (2006). HKL-3000: the integration of data reduction and structure solution—from diffraction images to an initial model in minutes. Acta Crystallographica Section D: Biological Crystallography 62, 859-866.
32. Emsley, P., Cowtan, K., and Iucr (2004). Coot: model-building tools for molecular graphics. Acta Crystallogr D Biol Crystallogr 60, 2126-2132.
33. PHENIX: a comprehensive Python-based system for macromolecular structure solution. P. D. Adams, P. V. Afonine, G. Bunkoczi, V. B. Chen, I. W. Davis, N. Echols, J. J. Headd, L. W. Hung, G. J. Kapral, R. W. Grosse-Kunstleve, A. J. McCoy, N. W. Moriarty, R. Oeffner, R. J. Read, D. C. Richardson, J. S. Richardson, T. C. Terwilliger, and P. H. Zwart. Acta Cryst. D66, 213-221 (2010).
34. Laskowski, R. A., MacArthur, M. W., Moss, D. S., and Thornton, J. M. (1993). PROCHECK: a program to check the stereochemical quality of protein structures. J Appl Crystallogr 26, 283-291.
35. Morin, A., Eisenbraun, B., Key, J., Sanschagrin, P. C., Timony, M. A., Ottaviano, M., and Sliz, P. (2013). Collaboration gets the most out of software. eLife Sciences 2, e01456.

What is claimed is:

1. A method of treating pain in a subject comprising administering to the subject one or more compounds selected from demeclocycline, chlortetracycline, and derivatives thereof.

2. The method of claim 1, wherein demeclocycline is administered to the subject.

3. The method of claim 1 or 2, wherein chlortetracycline is administered to the subject.

4. The method of any one of claims 1-3, further comprising administering minocycline or a derivative thereof to the subject.

5. The method of any one of claims 1-4, wherein the pain is chronic pain, acute pain, neuropathic pain, radicular pain associated with degenerative disc disease, diabetic neuropathy pain, complex regional pain syndrome, peripheral ischemic neuropathy pain, small fiber neuropathy pain, large fiber neuropathy pain, neuropathy associated with neurofibromatosis, post herpetic neuralgia pain, ilioinguinal neuralgia, anterior abdominal wall entrapment syndrome, lateral femerocutaneous neuralgia, cancer associated pain, somatic pain, visceral pain, or opioid withdrawal pain.

6. The method of any one of claims 1-5, wherein the compound prevents the pain.

7. The method of any one of claims 1-5, wherein the compound reduces the severity of the pain.

8. The method of any one of claims 1-7, wherein the one or more compounds are administered prior to surgery or prior to opioid cessation.

9. The method of any one of claims 1-8, wherein the one or more compounds are administered immediately after a surgical procedure or opioid cessation.

10. The method of any one of claims 1-9, wherein the method comprises administering to the subject at least two compounds selected from demeclocycline, chlortetracycline, minocycline, and derivatives thereof.

11. The method of claim 10, wherein the at least two compounds are administered substantially simultaneously.

12. The method of claim 10, wherein the at least two compounds are administered sequentially.

13. The method of any one of claims 10-12, wherein the IC₅₀ of the at least two compounds against an EphB enzyme in the subject is reduced relative to the IC₅₀ of a single compound of the at least two compounds.

14. The method of any one of claims 10-13, wherein an administered dose of the at least two compounds is reduced compared to a standard administered dose of a single compound of the at least two compounds.

15. The method of any one of claims 1-14, wherein the method comprises administering to the subject at least three compounds selected from demeclocycline, chlortetracycline, minocycline, and derivatives thereof.

16. The method of claim 15, wherein the at least three compounds are administered substantially simultaneously.

17. The method of claim 1516, wherein the at least three compounds are administered sequentially.

18. The method of any one of claims 15-17, wherein the IC₅₀ of the at least three compounds against an EphB enzyme in the subject is reduced relative to the IC₅₀ of a single compound of the at least three compounds.

19. The method of any one of claims 1-18, wherein the method comprises administering to the subject demeclocycline, chlortetracycline, and minocycline.

20. The method of claim 19, wherein the demeclocycline, chlortetracycline, and minocycline are administered substantially simultaneously.

21. The method of claim 19, wherein the demeclocycline, chlortetracycline, and minocycline are administered sequentially.

22. The method of any of claims 19-21, wherein the demeclocycline, chlortetracycline, and minocycline are administered in the same composition.

23. The method of any of claims 19-21, wherein the demeclocycline, chlortetracycline, and minocycline are administered in two or more compositions.

24. The method of any of claims 19-23, wherein the IC₅₀ of the demeclocycline, chlortetracycline, and minocycline against an EphB enzyme in the subject is reduced relative to the IC₅₀ of demeclocycline, chlortetracycline, or minocycline alone.

25. The method of any one of claims 1-24, further comprising administration of an additional therapeutic agent.

26. The method of claim 25, wherein the additional therapeutic agent is an analgesic.

27. The method of claim 26, wherein the analgesic is an opioid, a nonsteroidal anti-inflammatory drug (NSAID), acetaminophen, a steroid, a COX-2 inhibitor, a topical analgesic, or any combination thereof.

28. The method of claim 27, wherein the topical analgesic is lidocaine, capsaicin, or menthol.

29. The method of claim 25, wherein the additional therapeutic is an anticonvulsant.

30. The method of claim 29, wherein the anticonvulsant is a gabapentinoid.

31. The method of claim 25, wherein the additional therapeutic is an antidepressant.

32. The method of claim 31, wherein the antidepressant is a selective serotonin reuptake inhibitor.

33. The method of any one of claims 1-32, wherein the subject does not have a bacterial infection.

34. The method of any one of claims 1-33, wherein the method comprises inhibiting an Eph receptor in the subject with the compound.

35. The method of claim 34, wherein the compound binds to the catalytic domain of the Eph receptor.

36. The method of claim 34 or 35, wherein the compound does not disrupt an interaction between the Eph receptor and an ephrin ligand.

37. The method of any one of claims 34-36, wherein the compound is provided at a dose sufficient to reduce the activity of the Eph receptor by at least 50%.

38. The method of any one of claims 34-37, wherein the Eph receptor is EphB1, EphB2, EphB3, EphB4, or a variant thereof.

39. The method of any one of claims 34-38, wherein the Eph receptor is EphB1.

40. A pharmaceutical composition comprising:
(a) minocycline or a derivative thereof; and
(b) a compound selected from demeclocycline, chlortetracycline, and derivatives thereof.

41. The pharmaceutical composition of claim 40, wherein the pharmaceutical composition further comprises a pharmaceutically acceptable excipient.

42. The pharmaceutical composition of claim 41, wherein the pharmaceutical composition comprises an amount of the minocycline or derivative thereof and the compound sufficient to reduce the activity of an Eph receptor by at least 50%.

43. The pharmaceutical composition of claim 42, wherein the Eph receptor is EphB1, EphB2, EphB3, EphB4, or a variant thereof.

44. The pharmaceutical composition of claim 42 or 43, wherein the Eph receptor is EphB1.

45. The pharmaceutical composition of any of claims 40-44, wherein the pharmaceutical composition comprises:
(a) minocycline or a derivative thereof;
(b) demeclocycline or a derivative thereof; and
(c) chlortetracycline or a derivative thereof.

46. The pharmaceutical composition of any of claims 40-45, wherein the pharmaceutical composition comprises minocycline, demeclocycline, and chlortetracycline.

47. The pharmaceutical composition of any of claims 40-46, wherein the pharmaceutical composition further comprises an additional therapeutic agent.

48. The pharmaceutical composition of claim 47, wherein the additional therapeutic agent is an analgesic.

49. The method of claim 48, wherein the analgesic is an opioid, a nonsteroidal anti-inflammatory drug (NSAID), acetaminophen, a steroid, a COX-2 inhibitor, a topical analgesic, or any combination thereof.

50. The pharmaceutical composition of claim 49, wherein the topical analgesic is lidocaine, capsaicin, or menthol.

51. The pharmaceutical composition of claim 47, wherein the additional therapeutic is an anticonvulsant.

52. The pharmaceutical composition of claim 51, wherein the anticonvulsant is a gabapentinoid.

53. The pharmaceutical composition of claim 47, wherein the additional therapeutic is an antidepressant.

54. The pharmaceutical composition of claim 53, wherein the antidepressant is a selective serotonin reuptake inhibitor.

55. A kit comprising the pharmaceutical composition of any one of claims 40-54.

56. The kit of claim 55, wherein the kit further comprises instructions for use.

57. A method of treating an EphB receptor-associated condition in a subject comprising administering to the subject one or more compounds selected from demeclocycline, chlortetracycline, and derivatives thereof.

58. The method of claim 57, wherein the EphB receptor-associated condition is a cancer.

59. The method of claim 58, wherein the subject was previously treated for the cancer.

60. The method of claim 58, wherein the subject was determined to be resistant to the previous treatment.

61. The method of claim 58, wherein the cancer is a metastatic cancer.

60. The method of claim 58, wherein the cancer is a recurrent cancer.

61. The method of claim 58, wherein the cancer is a Stage I cancer.

62. The method of claim 58, wherein the cancer is a Stage II cancer.

63. The method of claim 58, wherein the cancer is a Stage III cancer.

64. The method of claim 58, wherein the cancer is a Stage IV cancer.

65. The method of claim 58, wherein the cancer is brain cancer.

66. The method of any of claim 65, wherein the cancer is a grade I cancer.

67. The method of any of claim 65, wherein the cancer is a grade II cancer.

68. The method of any of claim 65, wherein the cancer is a grade III cancer.

69. The method of any of claim 65, wherein the cancer is a grade IV cancer.

70. The method of any of claim 65, wherein the brain cancer is glioblastoma.

71. The method of any of claims 57-70, wherein demeclocycline is administered to the subject.

72. The method of any of claims 57-71, wherein chlortetracycline is administered to the subject.

73. The method of any of claims 57-72, further comprising administering minocycline or a derivative thereof to the subject.

74. The method of any of claims 57-73, wherein the method comprises administering to the subject at least two compounds selected from demeclocycline, chlortetracycline, minocycline, and derivatives thereof.

75. The method of claim 74, wherein the at least two compounds are administered substantially simultaneously.

76. The method of claim 74, wherein the at least two compounds are administered sequentially.

77. The method of any one of claims 57-76, wherein the IC₅₀ of the at least two compounds against an EphB enzyme in the subject is reduced relative to the IC₅₀ of a single compound of the at least two compounds.

78. The method of any one of claims 57-76, wherein an administered dose of the at least two compounds is reduced compared to a standard administered dose of a single compound of the at least two compounds.

79. The method of any one of claims 57-78, wherein the method comprises administering to the subject at least three compounds selected from demeclocycline, chlortetracycline, minocycline, and derivatives thereof.

80. The method of claim 79, wherein the at least three compounds are administered substantially simultaneously.

81. The method of claim 79, wherein the at least three compounds are administered sequentially.

82. The method of any one of claims 79-81, wherein the IC₅₀ of the at least three compounds against an EphB enzyme in the subject is reduced relative to the IC₅₀ of a single compound of the at least three compounds.

83. The method of any one of claims 57-82, wherein the method comprises administering to the subject demeclocycline, chlortetracycline, and minocycline.
